(19) United States
(12) Patent Application Publication
(10) Pub. No.: US 2024/0299647 A1
(43) Pub. Date: Sep. 12, 2024

(54) RESERVOIR ASSEMBLY AND DRUG SOLUTION INJECTION DEVICE COMPRISING SAME

(71) Applicant: EOFLOW CO, LTD., Seongnam-si (KR)

(72) Inventors: Yong Joon HAN, Seoul (KR); Seung Ha KIM, Goyang-si (KR); Sang Hyun HAN, Incheon (KR); Ki Ung KIM, Incheon (KR); Won Kyung BANG, Seoul (KR)

(21) Appl. No.: 18/666,763

(22) Filed: May 16, 2024

Related U.S. Application Data
(63) Continuation of application No. PCT/KR2022/017460, filed on Nov. 8, 2022.

(30) Foreign Application Priority Data
Nov. 16, 2021 (KR) 10-2021-0157427
Nov. 24, 2021 (KR) 10-2021-0162919
Dec. 7, 2021 (KR) 10-2021-0173421

(51) Int. Cl. A61M 5/142; A61M 5/145

(52) U.S. Cl. CPC A61M 5/14248; A61M 5/1452

(57) ABSTRACT

The present disclosure relates to a medical liquid infusion apparatus and a reservoir assembly. In detail, the reservoir assembly includes a reservoir that provides a storage space therein for the medical liquid and has an opening formed in a front side of the reservoir, and a plunger that is inserted into the reservoir and moves inside the reservoir to form a storage space for the medical liquid, wherein an amount of a stored medical liquid may be measured by the reservoir, the plunger, or a sensor unit disposed outside the reservoir and the plunger, and driving of the medical liquid infusion apparatus may be set based on a measured result.

### CROSS REFERENCE TO RELATED APPLICATIONS

This application is a Bypass Continuation of International Application No. PCT/KR2022/017460, filed on Nov. 8, 2022, and claims priority from and the benefit of Korean Patent Application No. 10-2021-0157427, filed on Nov. 16, 2021, Korean Patent Application No. 10-2021-0162919, filed on Nov. 24, 2021, and Korean Patent Application No. 10-2021-0173421, filed on Dec. 7, 2021, each of which is incorporated by reference for all purposes as if fully set forth herein.

### BACKGROUND

### Field

The present disclosure relates to a reservoir assembly and a medical liquid including the same.

### Discussion of the Background

Medical liquid infusion apparatuses such as insulin infusing apparatuses are used to infuse medical liquids into the bodies of patients. Such medical liquid infusion apparatuses are used by professional medical staff such as nurses or doctors but are mostly used by patients themselves or ordinary persons such as caretakers.

In the case of diabetic patients, particularly pediatric diabetic patients, it is necessary to infuse medical liquids such as insulin into a human body at regular intervals. A patch-type medical liquid infusion apparatus that may be used by being attached to a human body for a predetermined period of time has been developed, and such a medical liquid infusion apparatus may be used while being attached as a patch type to the human body such as the abdomen or waist of a patient for a predetermined period of time.

In order to increase the effect of the medical liquid infusion, the medical liquid infusion apparatus needs to be controlled to precisely infuse the medical liquid into the body of a patient, and accordingly, it is important to precisely infuse a small amount of the medical liquid through a small-sized medical liquid infusion apparatus.

The medical liquid infusion apparatus attached to the human body needs to be comfortable to wear, to be convenient to use, to be durable, and to be driven with low power. In particular, since a patient directly attaches the medical liquid infusion apparatus to the skin and uses it, it is important for a user to drive the medical liquid infusion apparatus conveniently and safely.

### SUMMARY

The present disclosure provides a reservoir assembly that may safely deliver a set amount of medical liquid and a medical liquid infusion apparatus including the same.

A reservoir assembly may include a reservoir that provides a space therein, a plunger that is inserted into the reservoir and moves back and forth in one direction of the reservoir to form a storage space for a medical liquid, a rod fixed to the plunger and extending in the one direction, a connecting member screw-coupled to the rod and movable in relation to the rod, and a pressing member disposed to generate a pushing force between the plunger and the connecting member when the plunger and the connecting member are adjacent to each other.

A medical liquid infusion apparatus and a reservoir assembly according to an embodiment of the present disclosure may store an accurate amount of a medical liquid in a reservoir and provide the medical liquid to a user. Before the medical liquid is infused into a user, the volume of the maximum storage space may be prevented from changing due to vibration applied to the medical liquid infusion apparatus and the reservoir assembly.

The medical liquid infusion apparatus and the reservoir assembly according to an embodiment of the present disclosure may measure an infusion amount of the medical liquid stored in the reservoir. A sensor unit may measure the amount of medical liquid stored in the reservoir, and the operation of the medical liquid infusion apparatus may be set based on the same. When a plunger moves linearly inside the reservoir, a connector connected to the plunger also moves to contact or release contact with the sensor unit, and the amount of the medical liquid stored in the reservoir may be sensed. The shape of the connector may be modified or processed to enhance stiffness and flexibility separately or simultaneously.

The medical liquid infusion apparatus and the reservoir assembly according to an embodiment of the present disclosure may prevent the plunger from moving from a set position before a user uses the medical liquid infusion apparatus, thereby storing a set amount of a medical liquid and supplying the medical liquid to the user. Also, it is possible to prevent bubbles from forming in the stored medical liquid. Of course, the scope of the present disclosure is not limited by these effects.

### BRIEF DESCRIPTION OF THE DRAWINGS

FIG. 1 is a block diagram illustrating a drug injection system according to an embodiment of the present disclosure.

FIG. 2 is a perspective view of a medical liquid infusion apparatus according to an embodiment of the present disclosure.

FIG. 3 is an exploded perspective view of a medical liquid infusion apparatus according to an embodiment of the present disclosure.

FIG. 4 is a diagram showing a reservoir assembly of FIG. 3.

FIG. 5 is a cross-sectional view taken along a line V-V′ of FIG. 2, according to an embodiment.

FIG. 6 is a diagram showing an initial state in which no medical liquid is stored in a storage space of the reservoir of FIG. 4.

FIG. 7 is an enlarged view of portion A of FIG. 6.

FIGS. 8 to 10 are cross-sectional views of an operation of infusing a medical liquid into the reservoir of FIG. 6, storing the medical liquid, and discharging the medical liquid through a needle.

FIG. 11 is a perspective view of a reservoir assembly according to another embodiment of the present disclosure.

FIG. 12 is an exploded perspective view of the reservoir assembly of FIG. 11.

FIGS. 13 to 15 are diagrams showing the operation of the reservoir assembly of FIG. 12.

FIG. 16 is an exploded perspective view of the reservoir assembly in FIG. 12, further including an auxiliary force member.

FIG. 17 is a cross-sectional view of the reservoir assembly of FIG. 16.

FIG. 18 is a side cross-sectional view of a reservoir assembly according to another embodiment of the present disclosure.

FIG. 19 is an enlarged view of a region B of FIG. 18.

FIG. 20 is a diagram illustrating a protrusion according to an embodiment different from that of FIG. 19.

FIG. 21 is a diagram illustrating a protrusion according to an embodiment different from that of FIG. 19.

FIG. 22 is a diagram showing a state in which a predetermined amount of a medical liquid is infused in FIG. 18.

FIG. 23 is a diagram showing a state in which the maximum amount of the medical liquid has been infused in FIG. 18.

FIG. 24 is a perspective view of a driving unit and a driving module, according to another embodiment of the present disclosure.

FIG. 25 is a diagram showing a reservoir assembly according to another embodiment of the present disclosure.

### DETAILED DESCRIPTION

According to an embodiment of the present disclosure, a reservoir assembly includes a reservoir that provides a space therein, a plunger that is inserted into the reservoir and moves back and forth in one direction of the reservoir to form a storage space for a medical liquid, a rod fixed to the plunger and extending in the one direction, a connecting member screw-coupled to the rod and movable in relation to the rod, and a pressing member disposed to generate a pushing force between the plunger and the connecting member when the plunger and the connecting member are adjacent to each other.

Also, screw thread are formed on an outer peripheral surface of the rod, screw threads are formed on an inner peripheral surface of the connecting member, and the rod may be screw-coupled to the connecting member by being inserted into the connecting member.

Also, screw threads may be formed in a predetermined region of the connecting member.

Also, the pressing member may be provided as a spring.

Also, the pressing member may surround a front end of the rod and extend toward the connecting member while a front end of the pressing member is fixed to the plunger.

According to another embodiment of the present disclosure, a medical liquid infusion apparatus includes a reservoir assembly in which a medical liquid is stored, a needle assembly fluidly connected to the reservoir assembly to discharge the medical liquid, and a driving unit connected to the reservoir assembly and configured to move the medical liquid from the reservoir to the needle assembly when driven, wherein the reservoir assembly includes a reservoir that provides a space therein, a plunger that is inserted into the reservoir and moves back and forth in one direction of the reservoir to form a storage space for a medical liquid, a rod fixed to the plunger and extending in a direction away from the storage space, a connecting member screw-coupled to the rod and movable in relation to the rod, and a pressing member disposed to generate a pushing force between the plunger and the connecting member when the plunger and the connecting member are adjacent to each other.

Since the present disclosure can apply various transformations and can have various embodiments, specific embodiments are illustrated in the drawings and described in detail in the detailed description. Effects and features of the present disclosure, and a method of achieving them will become clear with reference to the embodiments described below in detail in conjunction with the drawings. However, the present disclosure is not limited to the embodiments disclosed below and may be implemented in various forms.

Hereinafter, embodiments of the present disclosure will be described in detail with reference to the accompanying drawings, and when described with reference to the drawings, the same or corresponding components are given the same reference numerals, and overlapping descriptions thereof will be omitted.

In the following examples, the singular expression includes the plural expression unless the context clearly dictates otherwise.

In the following embodiments, terms such as include or have means that the features or components described in the specification are present, and the possibility that one or more other features or components will be added is not excluded in advance.

In cases where certain embodiments may be implemented otherwise, a specific process sequence may be performed differently from the described sequence. For example, two processes described in succession may be performed substantially simultaneously or may be performed in an order opposite to the order described.

In the drawings, the size of the components may be exaggerated or reduced for convenience of description. For example, since the size and thickness of each component shown in the drawings are arbitrarily indicated for convenience of description, embodiments below are not necessarily limited to those illustrated.

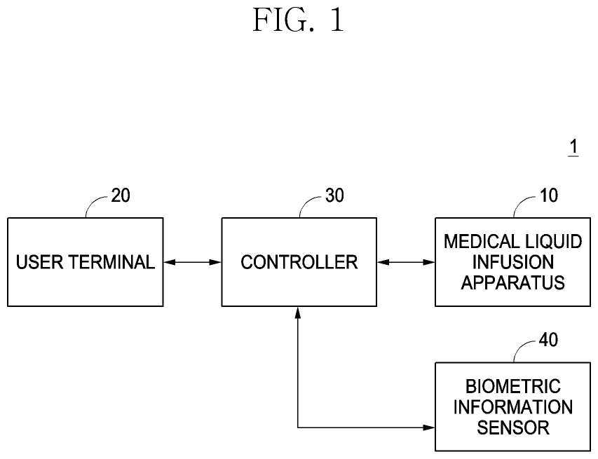

FIG. 1 is a block diagram illustrating a medical liquid infusion system 1 according to an embodiment of the present disclosure.

Referring to FIG. 1, the medical liquid infusion system 1 may include a medical liquid infusion apparatus 10, a user terminal 20, a controller 30 and a biometric information sensor 40. In the medical liquid infusion system 1, a user is capable of driving and controlling the medical liquid infusion system 1 by using the user terminal 20, and, based on blood glucose information monitored by the biometric information sensor 40, the medical liquid infusion apparatus 10 may periodically inject a medical liquid.

The medical liquid infusion apparatus 10 also performs a function of injecting a medical liquid that needs to be infused to a user, e.g., insulin, glucagon, anesthetics, painkillers, dopamine, growth hormone, and smoking cessation aid, based on data sensed by the biometric information sensor 40.

Also, the medical liquid infusion apparatus 10 may transmit a device status message including information regarding the remaining battery capacity of the medical liquid infusion apparatus 10, a result (success or failure) of booting of the medical liquid infusion apparatus 10, a result (success or failure) of injection, etc., to the controller 30. Messages transmitted to the controller 30 may be transmitted to the user terminal 20 via the controller 30. Alternatively, the controller 30 may transmit enhanced data obtained by processing received messages to the user terminal 20.

According to an embodiment, the medical liquid infusion apparatus 10 may be provided separately from the biometric information sensor 40 and may be installed to be spaced apart from a subject. According to another embodiment, the medical liquid infusion apparatus 10 and the biometric information sensor 40 may be provided within a single device.

According to an embodiment, the medical liquid infusion apparatus 10 may be mounted on a user's body. In addition, according to another embodiment, the medical liquid infusion apparatus 10 may also be mounted on an animal and may infuse a medical liquid thereto.

The user terminal 20 may receive an input signal from a user to drive and control the medical liquid infusion system 1. The user terminal 20 may generate a signal for driving the controller 30 and control the controller 30 to drive the medical liquid infusion apparatus 10. Also, the user terminal 20 may display biometric information measured by the biometric information sensor 40 and display status information regarding the medical liquid infusion apparatus 10.

The user terminal 20 refers to a communication terminal usable in a wired/wireless communication environment. For example, the user terminal 20 may include a smartphone, a tablet PC, a PC, a smart TV, a mobile phone, a personal digital assistant (PDA), a laptop computer, a media player, a micro server, a global positioning system (GPS) device, an e-book reader, a digital broadcasting terminal, a navigation device, a kiosk, an MP3 player, a digital camera, a home appliance, a device equipped with a camera, and other mobile or non-mobile computing devices. Also, the user terminal 20 may be a wearable device having a communication function and a data processing function, e.g., a watch, glasses, a hair band, and a ring. However, as described above, any terminal equipped with an application capable of internet communication may be employed without limitation.

The user terminal 20 may be connected one-to-one with a pre-registered controller 30. The user terminal 20 may be connected to the controller 30 through encryption to prevent the controller 30 from being driven and controlled by an external device.

According to an embodiment, the user terminal 20 and the controller 30 may be separated from each other and provided as separate devices. For example, the controller 30 may be provided to a subject equipped with the medical liquid infusion apparatus 10, and the user terminal 20 may be provided to the subject or a third person. The user terminal 20 may be driven by a guardian, and thus the safety of the medical liquid infusion system 1 may be improved.

According to another embodiment, the user terminal 20 and the controller 30 may be provided as a single device. The controller 30 integrated with the user terminal 20 may communicate with the medical liquid infusion apparatus 10 and control injection of a medical liquid.

The controller 30 may perform a function of transmitting and receiving data to and from the medical liquid infusion apparatus 10, transmit a control signal related to the injection of a medical liquid such as insulin to the medical liquid infusion apparatus 10, and receive a control signal related to the measurement of a biometric value such as blood glucose from the biometric information sensor 40.

For example, the controller 30 may transmit an instruction request to measure the current state of a user to the medical liquid infusion apparatus 10 and receive measurement data from the medical liquid infusion apparatus 10 in response to the instruction request.

The biometric information sensor 40 may perform a function of measuring a user's biometric values such as a blood glucose level, a blood pressure, a heart rate, etc. according to purposes. Data measured by the biometric information sensor 40 may be transmitted to the controller 30, and an infusion cycle and/or an infusion amount of a medical liquid may be set based on the measured data. Data measured by the biometric information sensor 40 may be transmitted to the user terminal 20 and displayed thereon.

According to an embodiment, the biometric information sensor 40 may be a sensor that measures a blood glucose level of a subject. The biometric information sensor 40 may be a continuous glucose monitoring (CGM) sensor. A CGM sensor may be attached to a subject to continuously monitor a blood glucose level of the subject.

The user terminal 20, the controller 30, and the medical liquid infusion apparatus may perform communication with one another by using a network. For example, the network is a comprehensive data communication network that includes a local area network (LAN), a wide area network (WAN), a value added network (VAN), a mobile radio communication network, a satellite communication network, and mutual combinations thereof and allows network constituent entities to communicate smoothly with one another and may include wired Internet, wireless Internet, and a mobile wireless communication network. Also, wireless communication may include, for example, wireless LAN (Wi-Fi), bluetooth, bluetooth low energy, Zigbee, Wi-Fi direct (WFD), ultra-wideband (UWB), infrared communication (IrDA, infrared data association), near field communication (NFC), and 5G, but the present disclosure is not limited thereto.

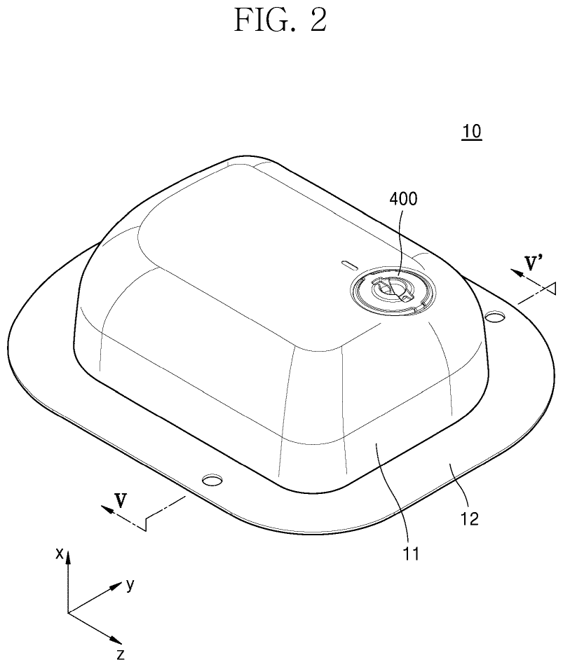

FIG. 2 is a perspective view of a medical liquid infusion apparatus according to an embodiment of the present disclosure.

Referring to FIG. 2, the medical liquid infusion apparatus 10 may be attached to a user subject to medical liquid infusion and may infuse a medical liquid stored therein to the user in a set amount.

The medical liquid infusion apparatus 10 may be used for various purposes depending on the type of medical liquid to be infused. For example, the medical liquid may include an insulin-based medical liquid for diabetic patients, other medical liquids for pancreas, cardiac medical liquids, and other various types of medical liquids.

The medical liquid infusion apparatus 10 according to an embodiment may include a housing 11 covering the outer portion and an attachment portion 12 positioned adjacent to the skin of a user. The medical liquid infusion apparatus 10 includes a plurality of parts arranged in an inner space between the housing 11 and the attachment portion 12. A separate bonding means may be further provided between the attachment portion 12 and the skin of a user, and the medical liquid infusion apparatus 10 may be fixed to the skin by the bonding means.

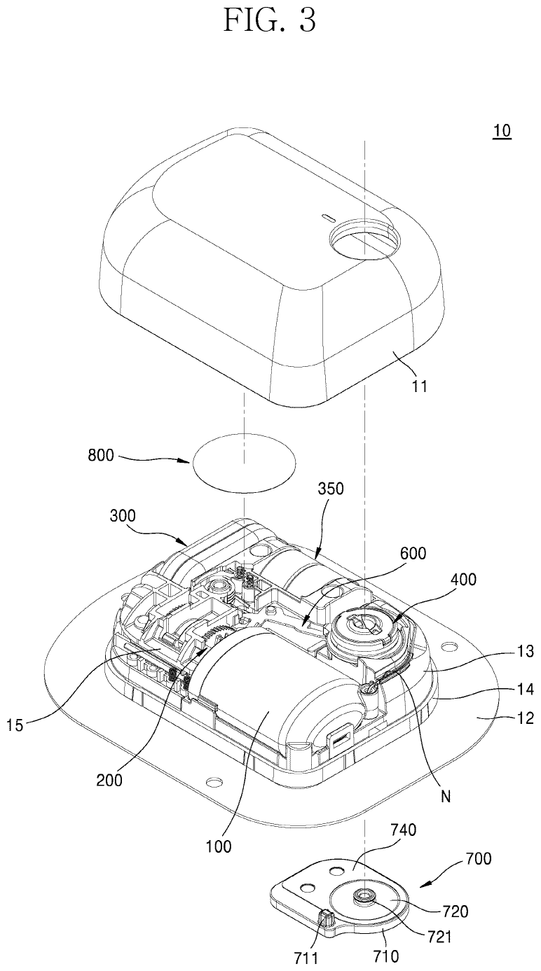

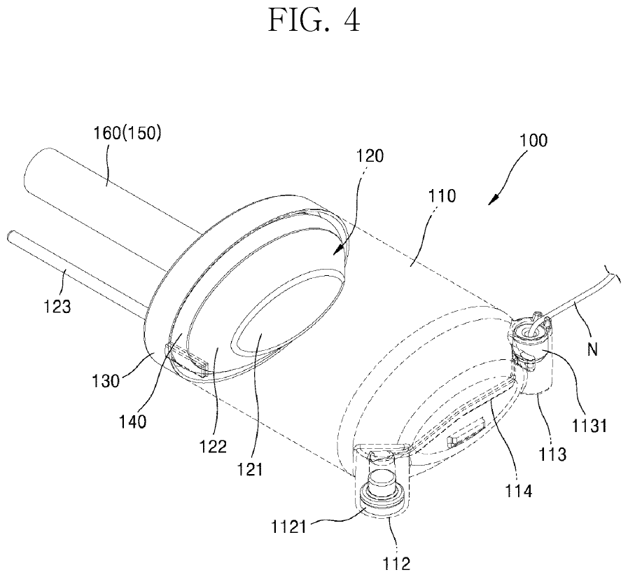

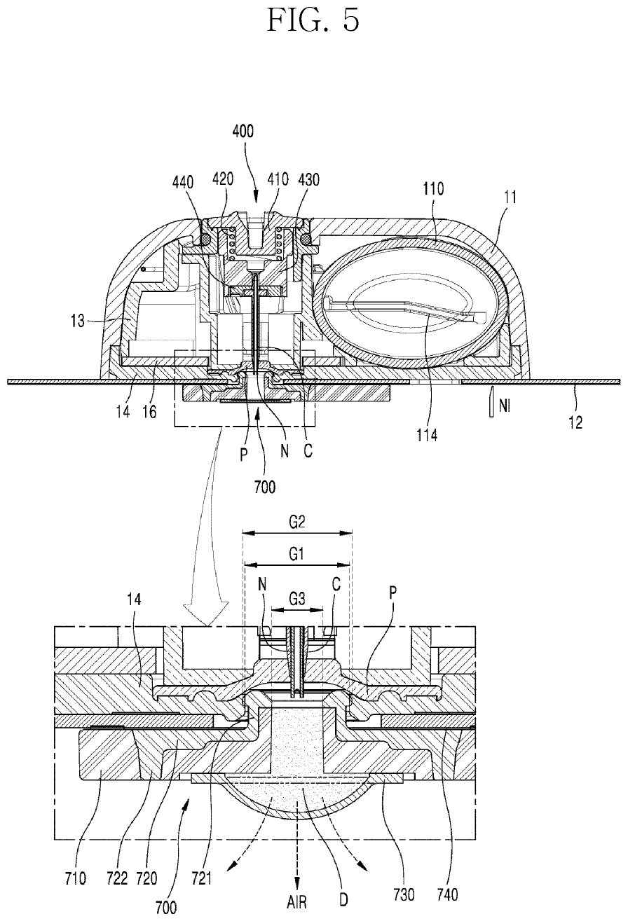

FIG. 3 is an exploded perspective view of a medical liquid infusion apparatus according to an embodiment of the present disclosure, FIG. 4 is a diagram showing a reservoir assembly of FIG. 3, and FIG. 5 is a cross-sectional view taken along a line V-V′ of FIG. 2, according to an embodiment.

Referring to FIG. 3, the medical liquid infusion apparatus 10 may include a reservoir assembly 100, a driving unit 200, a driving module 300, a battery 350, a needle assembly 400, a clutch unit 500, a trigger member 600, a needle cover assembly 700, and an alarm unit 800.

In the medical liquid infusion apparatus 10, a base body may form a frame in which at least one body supports internal components. The base body may include a first body 13, a second body 14, and a third body 15 according to arrangements.

The first body 13 is disposed below the housing 11 and may support the reservoir assembly 100, the driving module 300, the battery 350, the needle assembly 400, etc. in respective openings or grooves.

The second body 14 is disposed below the first body 13 and may be connected to the attachment portion 12. The second body 14 may cover the lower portion of the medical liquid infusion apparatus 10.

The third body 15 is disposed above the first body 13 and may support the reservoir assembly 100, the driving unit 200, the driving module 300, the battery 350, etc. in respective openings or grooves.

In the drawings, the first body 13, the second body 14 and the third body 15 are shown. However, the present disclosure is not limited thereto, and there may be a single integrated body or a plurality of bodies.

Referring to FIG. 4, the reservoir assembly 100 may store a medical liquid in a space formed inside a reservoir 110, and may move the medical liquid to a needle N in a set amount as a plunger 120 moves.

The reservoir assembly 100 may include the reservoir 110, the plunger 120, a cap cover 130, a contact member 123, a sealing ring 140, a rod 150, and a connecting member 160.

The reservoir 110 forms the outer portion of the reservoir assembly 100 and may provide an internal space in which at least portions of the plunger 120, the contact member 123, the rod 150, and the connecting member 160 may be accommodated.

The cross-section of the reservoir 110 in horizontal directions may have a longitudinal length and a latitudinal length different from each other. For example, the cross-section of the reservoir 110 may have an elliptical shape.

The reservoir 110 may extend to a predetermined length in the longitudinal direction and, in the internal space thereof, store a medical liquid in a storage space defined by the reservoir 110 and the plunger 120. As the medical liquid is stored in the reservoir 110, the plunger 120 may move rearward in the longitudinal direction of the reservoir 110. also, as the plunger 120 moves forward, the medical liquid may be discharged to the needle N.

The cap cover 130 is mounted on the rear end of the reservoir 110, and the contact member 123 and the rod 150 and/or the connecting member 160 may move through the cap cover 130 through an opening formed in the cap cover 130.

The reservoir 110 may have an inlet end 112 and an outlet end 113. A medical liquid may be infused through the inlet end 112 and discharged through the needle N installed at the outlet end 113.

The inlet end 112 is connected to the lower portion of a medical liquid infusion apparatus, and the medical liquid may be infused by a medical liquid infuser. A first sealing member 1121 is disposed at the inlet end 112, and the first sealing member 1121 may prevent leakage of a medical liquid.

The outlet end 113 is disposed to be apart from the inlet end 112 and is connected to the needle N, and thus a medical liquid may be discharged therethrough. A second sealing member 1131 is disposed at the outlet end 113, and the needle N may be fixed to the second sealing member 1131.

A guide groove 114 may be formed on an inner surface of the reservoir 110. At least a portion of the guide groove 114 may extend to interconnect the inlet end 112 and the outlet end 113.

The plunger 120 is inserted into the reservoir 110 and may reciprocate in one direction of the reservoir 110. According to an embodiment, the plunger 120 may move linearly within the reservoir 110 due to driving of the driving module 300 and the driving unit 200. When a medical liquid flows into the reservoir 110, the plunger 120 moves rearward, and, as the plunger 120 advances, the medical liquid stored in the storage space of the reservoir 110 may be discharged to the needle N.

The plunger 120 may have an end 121 and an inclined surface 122, at one end. The end 121 and the inclined surface 122 may form the front side of the plunger 120 and may be portions that define the storage space together with the reservoir 110. The end 121 and the inclined surface 122 may be in close contact with the inner surface of the reservoir 110 at the front end of the reservoir 110.

The plunger 120 may have the contact member 123 extending rearward. The contact member 123 is installed on the plunger 120 and may linearly move along with the linear movement of the plunger 120.

The contact member 123 includes a material having electrical conductivity and may have a shaft-like shape. As the contact member 123 moves and contacts a sensor unit, the storage amount of a medical liquid may be measured or the medical liquid infusion apparatus may be started to be driven.

The sealing ring 140 may be provided at a portion of the plunger 120 contacting the inner wall of the reservoir 110, thereby preventing leakage of a medical liquid stored in the storage space during the movement of the plunger 230.

Referring to FIG. 5, a control module 16 may be disposed inside the medical liquid infusion apparatus 10. The control module 16, which is a circuit board, is disposed below the second body 14 and may control the overall driving of the medical liquid infusion apparatus 10. The control module 16 may electrically contact the driving module 300, the battery 350, the alarm unit 800, and a plurality of sensor units (not shown) and control driving thereof.

Referring back to FIG. 3, the driving unit 200 may be installed between the driving module 300 and the reservoir assembly 100 and may move the plunger 120 placed within the reservoir 110 by using driving force generated by the driving module 300. However, the driving unit 200 may move the plunger 120 forward only when the rod 150 and a drive wheel are coupled or engaged to each other by the clutch unit 500.

The driving force generated by the driving module 300 may be transmitted to the driving unit 200. The driving force transmitted to the driving unit 200 may move the plunger 120 located at the rear of the reservoir 110 forward.

All kinds of devices with medical liquid suction power and medical liquid discharge power by electricity may be used as the driving module 300.

For example, all kinds of pumps such as mechanical displacement type micropumps and electromagnetic motion type micropumps may be used.

The mechanical displacement micro-pump is a pump that uses the motion of a solid like a gear or a diagram or a fluid to generate a pressure difference to induce a flow of a fluid and may include a diaphragm displacement pump, a fluid displacement pump, a rotary pump, etc.

The electromagnetic motion type micropump is a pump that directly uses electrical or magnetic energy to move a fluid, and includes an electro hydrodynamic pump (EHD), an electro osmotic pump, a magneto hydrodynamic pump, an electro wetting pump, and the like.

The battery 350 may supply electricity to the medical liquid infusion apparatus to activate components thereof. Although a pair of batteries are shown in the drawings, the present disclosure is not limited thereto, and the number of the batteries may be variously set according to the capacity, the use range, the use time, etc. of the medical liquid infusion apparatus.

The battery 350 is disposed adjacent to the driving module 300 and may supply electricity to the driving module 300. Also, the battery 350 is connected to the control module 16, and, based on an electrical signal measured by a sensor unit (not shown), the sensor unit may measure data regarding the number of rotations or the rotational speed of the driving unit 200, an amount of medical liquid stored in the reservoir 210, an amount of a medical liquid infused into a user, etc.

As shown in FIG. 5, the needle assembly 400 may be mounted on the first body 13. In the needle assembly 400, the needle N and/or a cannula C may move in an axial direction by rotation of a sleeve 410. The needle assembly 400 may include the sleeve 410, an elastic member 420, a first holder 430, a second holder 440, the needle N, the cannula C, and a patch P.

The sleeve 410 forms the outer portion of the needle assembly 400 and may be rotated around the central axis in the lengthwise direction. The elastic member 420 is disposed inside the sleeve 410, and thus the sleeve 410 may receive an expansion force from the elastic member 420.

The elastic member 420 may be disposed between the sleeve 410 and the first holder 430. When the elastic member 420 expands, the first holder 430 may be moved downward. Also, when the first holder 430 moves upward, the elastic member 420 may be compressed.

The first holder 430 may support the needle N. Since the needle N is inserted and fixed to one side of the first holder 430, when the first holder 430 moves in the axial direction, the needle N also moves. The first holder 430 is disposed in the inner space of the sleeve 410, while the elastic member 420 is disposed above the first holder 430.

The second holder 440 is disposed to face one side of the first holder 430 and may support the cannula C. The second holder 440 includes a flexible material, and thus the second holder 440 may be instantly deformed in shape when an external force is applied thereto. Alternatively, the second holder 440 may include a rigid material, and thus the second holder may be moved by a force applied by the first holder 430.

Since the needle N is fixed to the first holder 430, the needle N may be inserted into or released from the cannula C by the axial movement of the first holder 430. A first end of the needle N may be connected to a reservoir 410, and thus a medical liquid may be delivered through the needle N. Meanwhile, a second end of the needle N may be inserted into the cannula C, and thus the needle N may move along the cannula C.

Since the cannula C is fixed to the second holder 440, the cannula C may be inserted into the skin of a user by the axial movement of the second holder 440. Since the cannula C has a conduit shape capable of accommodating the needle N, a medical liquid discharged from the needle N may be infused into a user.

The patch P is supported on one side of the medical liquid infusion apparatus 10 and may fix the position of the cannula C. Since an end of the cannula C is supported by the patch P, it is possible to prevent the cannula C from being separated during storage or movement of the medical liquid infusion apparatus 10.

An end of the needle N and/or an end of the cannula C is inserted into the patch P. Before a medical liquid is infused into a user, that is, before the needle N and the cannula Care inserted into the user, the end of the needle N and/or the end of the cannula C may be supported by the patch P and positions thereof may be set. When a medical liquid is injected into the reservoir 110, the positions of the needle N and the cannula C are fixed, and thus gas remaining in the reservoir 110 may be stably discharged.

The cannula C maintains the state of being inserted into the skin of a user, but the needle N rises and is separated from the user. However, the cannula C and the needle N form a path through which a fluid is moved, and thus a medical liquid injected from the reservoir 110 may be infused into the user through the needle N and the cannula C.

In the medical liquid infusion apparatus 10, a user may simply rotate the needle assembly 400 to insert the cannula C into a subject and initiate medical liquid infusion. In the medical liquid infusion apparatus 10, as a user first rotates the sleeve 410, the cannula C may be inserted into a subject, and a knob (not shown) of the needle assembly 400 may press the trigger member 600 to drive the driving module 300.

According to an embodiment, the trigger member 600 may generate a mechanical signal for infusing a medical liquid of the medical liquid infusion apparatus 10. The trigger member 600 may be rotatably disposed on one side of the third body 15. The trigger member 600 rotates to start driving the driving module 300, and, at the same time, the clutch unit 500 may drivingly connect the driving unit 200.

In detail, when a user rotates the needle assembly 400, the knob (not shown) of the needle assembly 400 applies a force to the end of the trigger member 600, and thus rotation of the trigger member 600 may be initiated. As the trigger member 600 rotates, pressure is applied to an end of a coupler to be described later, and the coupler may be coupled to the connecting member 160 to activate the clutch unit 500 (not shown).

The needle cover assembly 700 may have a first cover 710, a second cover 720, a filter member 730, and an adhesive layer 740.

The first cover 710 may be disposed under the medical liquid infusion apparatus 10. The second cover 720 may be inserted and assembled into an opening of the first cover 710.

The second cover 720 is assembled to the first cover 710, and the needle N and/or the cannula C may be aligned to the center of the second cover 720. The second cover 720 may have a storage space which penetrates at the center in the heightwise direction and in which the medical liquid D is stored.

The first cover 710 has greater rigidity than the second cover 720. The first cover is a portion exposed to the outside and includes a material with a slightly greater rigidity than that of the second cover 720. The second cover 720 is assembled to the first cover 710 and includes a material having less rigidity than the first cover 710 to be inserted into an opening of the third body 15.

A protrusion 721 to be inserted into the second body 14 may be provided at the center of the second cover 720. Also, the second cover 720 has a fixing protrusion 722, and, as the fixing protrusion 722 is inserted into the first cover 710, the first cover 710 and the second cover 720 may be assembled.

The protrusion 721 of the second cover 720 is inserted into an opening at the bottom of the second body 14. A diameter G2 of the protrusion 721 is set to be slightly greater than a diameter G1 of the opening. Since the second cover 720 has a certain elasticity, the protrusion 721 may be inserted into the opening and fixed.

The second cover 720 has an inner diameter G3, and thus the needle N and the cannula C may be aligned to the top of the second cover 720. The inner diameter G3 forms a storage space of the second cover 720, and a medical liquid may be stored therein or a gas may be moved and discharged therefrom.

The filter member 730 is attached to the second cover 720. The filter member 730 is disposed under the storage space of the second cover 720, and a gas like air passes through the filter member 730, but a liquid like a medical liquid does not pass through the filter member 730. Therefore, the air discharged from the needle N passes through the filter member 730 and is discharged to the outside, but a medical liquid D discharged from the needle may be stored in the storage space defined by the second cover 720 and the filter member 730.

The shape of the filter member 730 may change according to the amount of the medical liquid D stored in the storage space. For example, when the storage space is filled with the medical liquid D, the filter member 730 expands downward, and thus a user may recognize that the medical liquid D has flowed into the needle cover assembly 700.

The adhesive layer 740 is disposed on one surface of the needle cover assembly and may attach the needle cover assembly 700 to the attachment portion 12.

The alarm unit 800 is disposed inside or outside the medical liquid infusion apparatus 10 and may notify a user of normal operation or malfunction of the medical liquid infusion apparatus 10.

For example, the alarm unit 800 is disposed below the housing 11 and is connected to a circuit board. The alarm unit 800 may generate a warning sound or emit light to deliver an alarm to a user.

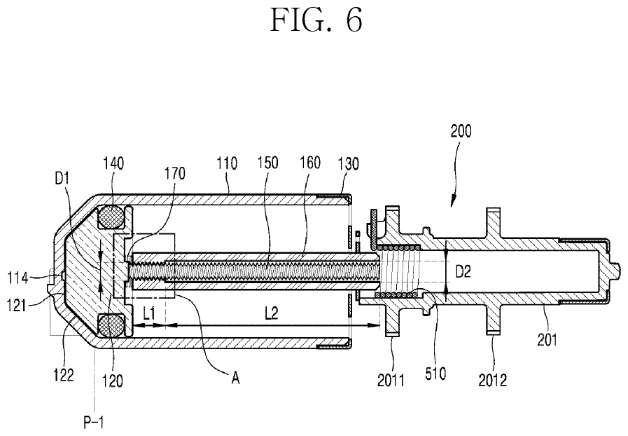

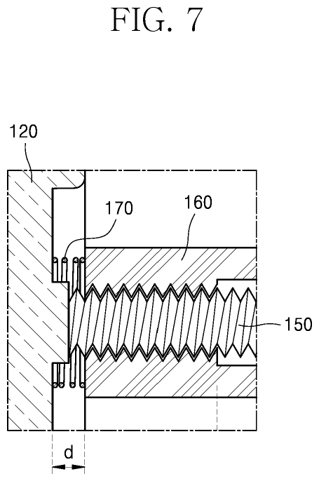

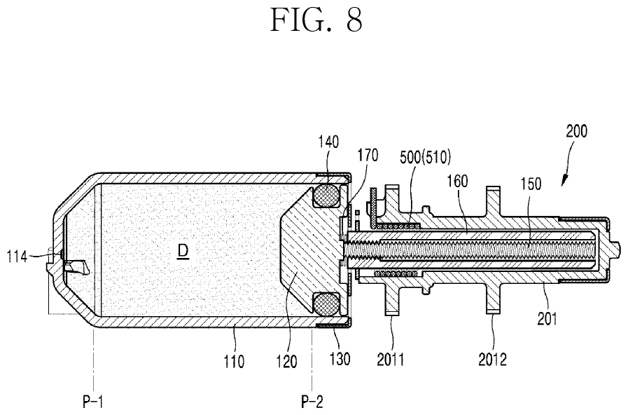

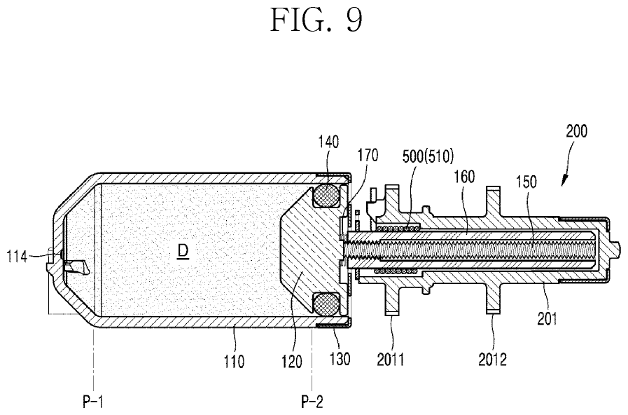

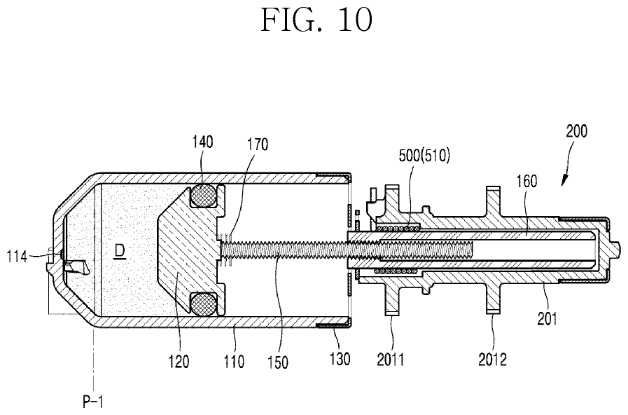

FIG. 6 is a diagram showing a state in which no medical liquid is stored in the storage space of the reservoir 110 of FIG. 4, FIG. 7 is an enlarged view of portion A of FIG. 6, FIG. 8 is a diagram showing a state in which a medical liquid is maximally stored in the storage space of the reservoir 100, FIG. 9 is a diagram showing a state in which the clutch unit 500 is activated in FIG. 8, and FIG. 10 is a diagram showing a state in which a medical liquid is being infused.

Referring to FIG. 6, the rod 150 may be fixed to the plunger 120 and extend rearward. The rod 150 may penetrate through the cap cover 130 through an opening of the cap cover 130 and move back and forth in one direction of the reservoir 110, i.e., the longitudinal direction of the reservoir 110, and at least a portion of the rod 150 may protrude to the outside of the reservoir 110.

The rod 150 is coupled to the connecting member 160, and thus, as the plunger moves, the rod 150 and the connecting member 160 may move together. Also, the rod 150 may move in relation to the connecting member 160. According to an embodiment, the rod 150 may have threads formed on the outer peripheral surface thereof in the longitudinal direction, and the connecting member 160 may have threads formed on the inner peripheral surface toward which the rod 150 is inserted. The rod 150 may be inserted into the connecting member 160 and screw-coupled thereto. In other words, since the rod 150 has a male thread and the connecting member 160 has a female thread, the rod 150 and the connecting member 160 may be screw-coupled to each other.

As the plunger 120 moves, while the rod 150 is being inserted into the connecting member 160 and screwed thereto, the rod 150 and the connecting member 160 may move together. Also, as the connecting member 160 rotates, the rod 150 may move in relation to the connecting member 160. In other words, in the reservoir assembly 100, the rotational movement of the connecting member 160 may be converted into the linear movement of the rod 150.

According to an embodiment, the connecting member 160 may have threads formed in a predetermined area of the connecting member 160. For example, a screw thread may be formed at one end of the connecting member 160, but no screw thread may be formed at the other end of the connecting member 160. A screw thread is formed on the inner circumferential surface of a first section L1 of the connecting member 160, and the rod 150 may be screw-coupled only in the first section L1. Also, the diameter of the first section L1 may be D1 in correspondence to that of the rod 150.

No screw thread may be formed on the inner circumferential surface of a second section L2 of the connecting member 160. Also, a diameter D2 of the second section L2 may be set to be greater than the diameter D1 of the first section L1. In the second section L2, the connecting member 160 may not contact the rod 150.

The length of the first section L1 may be set, such that the first section L1 overlaps the coupler 510 when the connecting member 160 moves backward. Referring to FIGS. and 9, when the plunger 120 extends to the rearmost position, at least a portion of the first section L1 may be disposed to overlap the coupler 510 (i.e., at least a portion of the first section L1 faces the coupler 510).

As shown in FIG. 9, when the clutch unit 500 is activated, the length of the first section L1 in the connecting member 160 may be set, such that the coupler 510 grips at least a portion of the first section L1. Since the rod 150 is screw-coupled only in the first section L1 of the connecting member 160, when the connecting member 160 rotates to move the rod 150 forward, the load due to the screw-coupling between the connecting member 160 and the rod 150 may be reduced.

When the clutch unit 500 is activated, the connecting member 160 may be rotated by the drive wheel 201. According to an embodiment, the drive wheel 201 is drivingly connected to the driving module 300 and may rotate by driving the driving module 300. The drive wheel may have a first connection end 2011 and a second connection end 2012 and may have an inner space in which the connecting member 160 may move. Since at least one of the first connection end 2011 and the second connection end 2012 is always drivingly connected to the driving module 300, the drive wheel 201 may rotate by driving of the driving module 300. For example, the first connection end 2011 and the second connection end 2012 may each have a gear teeth-like shape. As a connector (not shown) connected to the driving module 300 applies force to gear teeth, the drive wheel 201 may rotate.

When the clutch unit 500 is activated, the drive wheel 201 and the connecting member 160 may be drivingly connected to each other.

The clutch unit 500 may include the coupler 510, and the coupler 510 may be disposed between the rod 150 and the drive wheel 201. The coupler 510 is disposed outside the connecting member 160, is spaced apart from the connecting member 160 by a predetermined distance when the clutch unit 500 is deactivated, and, when the clutch unit 500 is activated, may be coupled to the connecting member 160 and connect the rod 150 and the drive wheel 201 to each other.

The coupler 510 is a component capable of applying an elastic force to the outside of the connecting member 160 and, for example, may have a spring-like shape.

When the drive wheel 201 and the connecting member 160 are engaged with each other by the coupler 510, by driving force generated by the driving module 300, the drive wheel of the driving unit 200 may rotate, and the connecting member 160 is rotated by the rotation of the drive wheel 201. As a result, the rod 150 may linearly move and move the plunger 120.

Referring to FIGS. 6 and 7, the reservoir assembly 100 may include a pressing member 170.

The pressing member 170 may be disposed between the plunger 120 and the connecting member 160. The pressing member 170 may press the plunger 120 forward and the connecting member 160 backward when the plunger 120 and the connecting member 160 are adjacent to each other. The pressing member 170 may be a component capable of generating a pushing force between the plunger 120 and the connecting member 160 when the plunger 120 and the connecting member 160 are adjacent to each other. For example, the pressing member may be a spring capable of applying an elastic force between the plunger 120 and the connecting member 160 in directions respectively opposite to the plunger 120 and the connecting member 160.

The pressing member 170 may be disposed, such that, while the front end of the pressing member 170 is fixed to the plunger 120, the pressing member 170 surrounds the rod 150 at the front end of the rod 150 or surrounds an engaged portion between the plunger 120 and the rod 150 and the rear end of the pressing member 170 faces toward the connecting member 160. At this time, the inner peripheral surface of the pressing member 170 and the outer peripheral surface of the rod 150 may be spaced apart from each other.

When the plunger 120 and the connecting member 160 are adjacent to each other, the rear end of the pressing member 170 may be in contact with the connecting member 160 and apply a force acting backward to the connecting member 160. The normal force acting between the rod 150 and adjacent threads of the connecting member 160 may increase due to the force applied to the connecting member 160. Therefore, the friction between the adjacent screw threads of the rod 150 and the connecting member 160 may increase, thereby preventing the screw-coupling between the rod 150 and the connecting member 160 from loosening.

In other words, when the plunger 120 and the connecting member 160 are adjacent to each other and a pushing force is generated between the plunger 120 and the connecting member 160 by the pressing member 170, firm screw-coupling may be formed between the rod 150 and the connecting member 160.

Therefore, until a medical liquid is infused to a user, a predetermined distance d may be constantly maintained between the plunger 120 and the connecting member 160, and the maximum volume of the storage space may be constantly maintained.

For example, as shown in FIG. 6, the plunger 120 may be positioned at the foremost position P-1 before a medical liquid is infused. At this time, the predetermined distance d may be maintained between the plunger 120 and the connecting member 160.

As a medical liquid flows into the reservoir 110 and the plunger 120 moves rearward, a storage space may be formed, and the volume of the storage space may gradually increase.

As shown in FIG. 8, when the plunger 120 is located at the rearmost position P-2, the volume of the storage space may be maximized. While the plunger 120 moves from the foremost position P-1 of the plunger 120 to the rearmost position P-2 of the plunger 120, the pressing member 170 may generate pushing force between the plunger 120 and the connecting member 160. The force generated by the pressing member 170 may strengthen the screw-coupling between the rod 150 and the connecting member 160, thereby preventing the predetermined distance d between the plunger 120 and the connecting member 160 from changing.

As the predetermined distance d between the plunger 120 and the connecting member 160 is maintained constant, the maximum volume of the storage space formed when the plunger 120 is located at the rearmost position P-2 may be maintained constant. By maintaining the maximum volume of the storage space constant, a set amount of a medical liquid may be infused into a user.

For example, when the screw-coupling between the rod 150 and the connecting member 160 is not strong enough and the distance between the plunger 120 and the connecting member 160 becomes longer than the predetermined distance d, in a state where all the medical liquid flowed into the reservoir 110, the plunger 120 may be at a position in front of the rearmost position P-2. This means that the maximum volume of the storage space is reduced and a smaller amount of a medical liquid is infused into a user.

Alternatively, when the distance between the plunger 120 and the connecting member 160 becomes shorter than the predetermined distance d, in a state where all the medical liquid flowed into the reservoir 110, the plunger 120 may be at a position beyond the rearmost position P-2. This means that the maximum volume of the storage space increases and a greater amount of a medical liquid is infused into a user.

The pressing member 170 may strengthen the screw-coupling between the rod and the connecting member 160 between the plunger 120 and the connecting member 160, thereby storing a set amount of a medical liquid in the reservoir 110 and supplying a set amount of the medical liquid to a user.

Referring to FIGS. 6 and 8 to 10, a process of storing a medical liquid in the reservoir 110 before a medical liquid infusion apparatus is attached to a user and infusing the medical liquid into the user by discharging the medical liquid from the reservoir 110 to a needle will be described.

### <Medical Liquid Storage Stage>

A user may infuse a medical liquid into the reservoir unit 200 of the medical liquid infusion apparatus by using an external medical liquid infuser (not shown). Referring to FIG. 6, before injection of a medical liquid, the plunger 120 is disposed at the front end of the reservoir 110, and the rod 150 is assembled to the connecting member 160 at the rear end of the plunger 120. At this time, since a clutch unit is in a deactivated state, the coupler 510 does not grip the connecting member 160, and thus the drive wheel 201 is not connected to the rod 150.

When a medical liquid begins to flow from the medical liquid infuser into the reservoir 110, the medical liquid may flow between the inner surface of the reservoir 110 and the plunger 120 and push the plunger 120 backward. As the plunger 120 moves backward, a storage space is formed between the reservoir 110 and the plunger 120, and, as the medical liquid flows in, the size of the storage space may gradually increase. When the plunger 120 moves backward and reaches the rearmost position P-2 at which the plunger 120 may not further move, the size of the storage space becomes maximum, and the medical liquid may no longer flow in.

### <Attachment Stage>

As shown in FIG. 8, when all the medical liquid D is stored in the reservoir 110, the medical liquid infusion apparatus may be attached to a user.

The user may attach the medical liquid infusion apparatus 10 to himself/herself and rotate the needle assembly 400, thereby inserting a needle and a cannula into the skin. The needle is inserted into the skin together with the cannula and may lead the cannula to be inserted into the skin.

Thereafter, the needle is withdrawn from the skin, but remains connected to the cannula. When the user further rotates the needle assembly 400, the needle moves upward while the cannula is inserted into the skin. The cannula and the needle are at least partially connected to each other and form and maintain a path through which the medical liquid D moves.

The pressing member 170 may maintain firm screw-coupling between the rod 150 and the connecting member 160 between the plunger 120 and the connecting member 160 while the plunger 120 moves backward and is positioned at the rearmost position P-2 due to introduction of the medical liquid D. Therefore, the relative position of the plunger 120 with respect to the connecting member 160 may be maintained.

### <Medical Liquid Infusion Stage>

The driving module 300 and the driving unit 200 are driven at substantially the same time as the cannula and the needle are inserted into the user. The medical liquid infusion apparatus may infuse the medical liquid D into the user according to a set cycle and a set infusion amount.

When the user rotates the needle assembly 400 to insert the needle and the cannula into the skin, the trigger unit may drive the driving module 300. When the driving module 300 is driven, a connector (not shown) connected to the driving module 300 may rotate around a rotation axis and rotate the drive wheel 201. The connector (not shown) may rotate the drive wheel 201 tooth-by-tooth while alternately pressing the first connection end 2011 and the second connection end 2012.

When the user rotates the needle assembly, the trigger unit 600 may activate the clutch unit 500. As shown in FIG. 9, when the clutch unit 500 is activated and the coupler 510 grips the outer portion of the connecting member 160, the drive wheel 201, the coupler 510, and the connecting member 160 may be integrated into one body. Therefore, when the drive wheel rotates, the connecting member 160 also rotates, and the rod 150 may move forward due to the rotation of the connecting member 160.

As shown in FIG. 10, as the rod 150 moves forward, the plunger 120 may also move forward. Since the connecting member 160 is being gripped by the coupler 510, the connecting member 160 may rotate while being fixed with respect to the longitudinal direction of the reservoir 110.

During rotation of the connecting member 160 while being fixed with respect to the longitudinal direction, the rod 150 and the plunger 120 move forward, and thus the plunger and the connecting member 160 are gradually spaced apart from each other and the force from the pressing member 170 may not act between the plunger 120 and the connecting member 160.

Forward movement of the plunger 120 may discharge the medical liquid D into the needle N. Therefore, the medical liquid D may be infused into the user according to a set driving cycle and a set driving speed of the driving module 300.

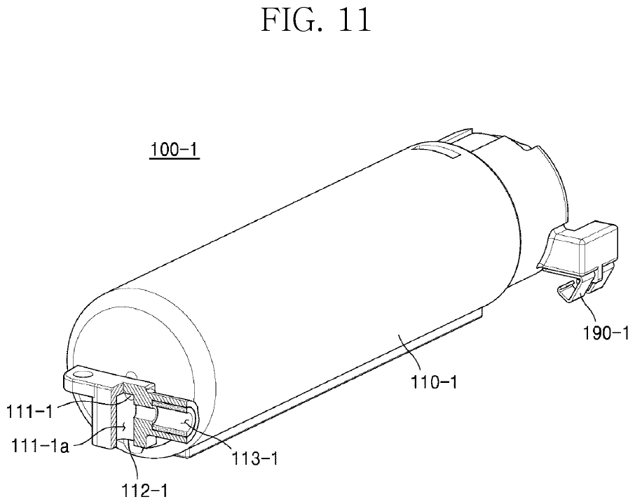

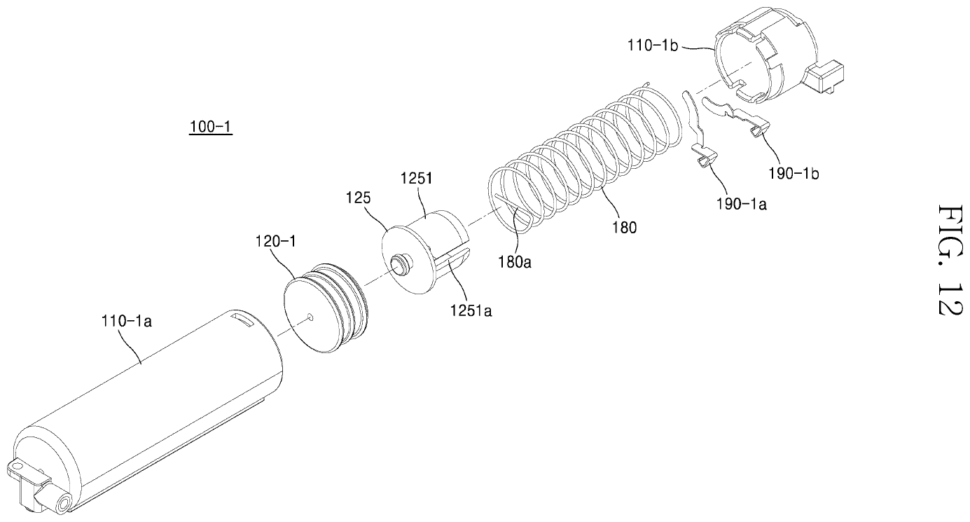

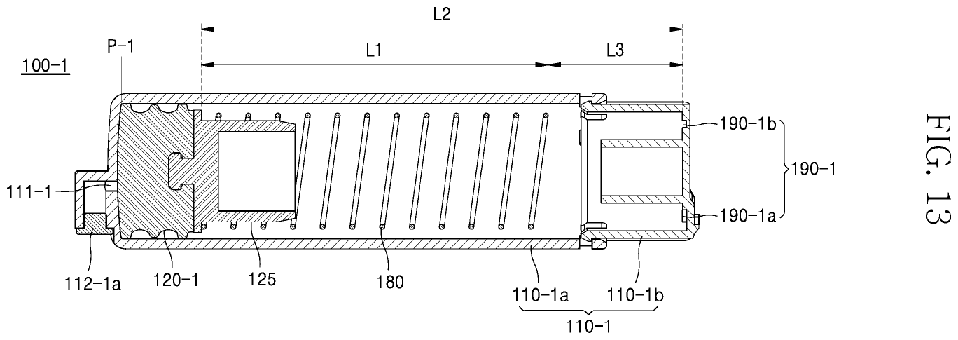

FIG. 11 is a perspective view of a reservoir assembly 100-1 according to another embodiment of the present disclosure, FIG. 12 is an exploded perspective view of the reservoir assembly 100-1 of FIG. 11, and FIGS. 13 to 15 are diagrams illustrating the operation of the reservoir assembly 100-1 of FIG. 11.

The reservoir assembly 100-1 stores a medical liquid in at least a portion of the internal space thereof and may discharge a set amount of the medical liquid into the needle assembly 400 by using driving force generated by operations of a driving unit and a driving module to be described later.

Referring to FIGS. 11 to 12, the reservoir assembly 100-1 may include a reservoir 110-1, a plunger 120-1, a connector 180, and a sensor unit 190-1.

The reservoir 110-1 forms the outer portion of the reservoir assembly 100-1, and may provide an internal space in which the plunger 120-1, connector 180, and sensor unit 190-1 may be accommodated.

For example, the reservoir 110-1 may have a cylindrical shape. In one embodiment, the reservoir 110-1 may be formed in a cylindrical shape, wherein the front portion and the rear portion may be closed and the reservoir 110-1 may be formed as a single body.

According to another embodiment, the reservoir 110-1 may include a reservoir case 110-1a having a closed front portion and an opened rear portion and a reservoir cap 110-1b that may be coupled to the rear portion of the reservoir case 110-1a. When the reservoir case 110-1a and the reservoir cap 110-1b are coupled to each other, an internal space in which the plunger 120-1, the connector 180, and the sensor unit 190-1 may be accommodated may be provided inside the reservoir case 110-1a and the reservoir cap 110-1b.

A portion of the internal space of the reservoir 110-1 is formed as a storage space for a medical liquid, and the storage space may be defined by the reservoir 110-1 and the plunger 120-1.

The reservoir 110-1 may have an opening 111-1 formed at the front side. The opening 111-1 may communicate with a flow path 111-1a formed in front of the reservoir 110-1. The flow path 111-1a may have an inlet end 112-1 formed at one end and an outlet end 113-1 formed at the other end. The opening 111-1 may communicate with the inlet end 112-1 through at least a portion of the flow path 111-1a and may communicate with the outlet end 113-1 through at least a portion of the flow path 111-1a.

A conduit (not shown) is connected to the outlet end 113-1, and the outlet end 113-1 may be connected to the driving unit through the conduit (not shown). The medical liquid stored in the storage space may sequentially pass through the opening 111-1, the flow path 111-1a, the outlet end 113-1, and the conduit (not shown) and flow into the needle assembly 400. A packing member 112-1a is provided at the inlet end 112-1 to prevent the medical liquid stored in the storage space from leaking out through the inlet end 112-1, and an infusion needle (not shown) may be inserted through the packing member 112-1a to infuse the medical liquid. The medical liquid introduced through the inlet end 112-1 may flow into the storage space through the flow path 111-1a and the opening 111-1.

The packing member 112-1a may have reclosable properties. For example, even when the infusion needle (not shown) penetrates through the packing member 112-1a and then is removed to fill the reservoir 110-1 with a medical liquid, the packing member 112-1a may prevent the medical liquid from leaking out through a portion of the packing member 112-1a penetrated by the infusion needle (not shown).

The packing member 112-1a may include a material containing polypropylene, thermoplastic elastomer, vegetable oil, etc.

The plunger 120-1 may be inserted into the reservoir 110-1, and the storage space may be defined in the internal space of the reservoir 110-1 by the reservoir 110-1 and the front side of the plunger 120-1.

The outer peripheral surface of the plunger 120-1 is in close contact with the inner peripheral surface of the reservoir 110-1 to prevent the medical liquid stored in the storage space from leaking out. For example, the plunger 120-1 may include an elastic material such as rubber or silicon and may be in close contact with the inner peripheral surface of the reservoir 110-1.

The front side of the plunger 120-1 forms a storage space for a medical liquid together with the reservoir 110-1, and, when the medical liquid is stored in the reservoir 110-1, the front side of the plunger 120-1 may contact the medical liquid.

The rear side of the plunger 120-1 may be connected to the connector 180.

According to an embodiment, the connector 180 may be installed directly at the rear side of the plunger 120-1.

According to another embodiment, the plunger 120-1 and the connector 180 may be connected to each other through a plunger holder 125. For example, the plunger holder 125 that is detachable or fixedly installed to the plunger 120-1 may be coupled to the rear of the plunger 120-1. A connector fixing portion 1251, which has a hollow structure and extends by a predetermined length, is formed at the rear of the plunger holder 125, and the connector 180 may surround the outer periphery of the connector fixing portion 1251. At this time, as a hook portion 180a formed in front of the connector 180 is inserted and fixed into the connector fixing portion through a fastening slit 1251a formed in at least a portion of the connector fixing portion 1251, the coupling between the plunger holder 125 and the connector 180 may be strengthened. For example, the fastening slit 1251a is formed in the connector fixing portion 1251 in a shape having the bent front end (an ‘L’-like shape), and thus the plunger holder 125 and the connector may be coupled to each other while the hook portion 180a is fixed at the bent portion of the fastening slit 1251a. The front side of the plunger holder 125 is coupled to the plunger 120-1, and the rear side of the plunger holder 125 is coupled to the connector 180, and thus the plunger holder 125 may connect the plunger 120-1 and the connector 180 to each other.

As the plunger 120-1 and the connector 180 are connected to each other, the connector 180 may move together with the plunger 120-1.

The connector 180 may extend to the rear side of the reservoir 110-1. The connector 180 is not limited to a specific shape and may have various shapes. For example, the connector 180 may be a coiled spring or a spiral spring. However, for convenience of explanation, descriptions below will focus on an embodiment in which the connector 180 is provided as a coiled spring as shown in the drawings.

The shape of the connector 180 may be modified or processed to enhance stiffness and flexibility separately or simultaneously.

The connector 180 may have flexibility and elasticity. The connector 180 may be compressed and restored by external force. When the plunger 120-1 moves rearward, the connector 180 also moves. However, when the rear side of the connector 180 is pressed, the connector 180 may be compressed and the plunger 120-1 may continue to move a predetermined distance. Then, when the plunger 120-1 moves forward, the connector 180 may be restored to its original state and move together with the plunger 120-1.

At least a portion of the connector 180 may include a conductive material. According to an embodiment, the connector 180 may include a metal material. The connector may be formed as a metal spring and may have certain stiffness and certain flexibility. According to another embodiment, a conductive material may be included in at least an outer region of the connector 180 having stiffness and flexibility. Since the connector 180 is conductive, when the connector 180 comes into contact with the sensor unit 190-1 provided at the rear side of the reservoir 110-1, the connector 180 may conduct electricity with the sensor unit 190-1.

The number of windings of the connector 180 may increase at the front portion and the rear portion of the connector 180 or the front end and the rear end of the connector 180 may be polished, and thus the connector 180 may be advantageous for connection to the plunger holder 125 and contact with the sensor unit 190-1.

The sensor unit 190-1 may be provided at the rear side of the reservoir 110-1.

The sensor unit 190-1 is a component for detecting medical liquid infusion into a storage space and may transmit a signal generated by the movement of the plunger 120-1 inside the reservoir 110-1 to the controller 30. The sensor unit 190-1 may be connected to a flexible printed circuit board (FPCB), and thus the sensor unit 190-1 may be electrically connected to the controller 30.

The sensor unit 190-1 may be provided as a variety of sensors capable of sensing the position of a component, such as an optical sensor or a contact sensor.

According to an embodiment, the sensor unit 190-1 may be provided as a contact sensor configured to measure the position of the plunger 120-1 based on a contact with the connector 180.

The sensor unit 190-1 may have a plurality of contact terminals. For example, the sensor unit 190-1 may include a first terminal 190-1a and a second terminal 190-1b.

The first terminal 190-1a and the second terminal 190-1b may be maintained spaced apart from each other, thereby forming an open circuit with respect to the sensor unit 190-1. The connector 180 may contact the first terminal 190-1a and the second terminal 190-1b and short the first terminal 190-1a and the second terminal 190-1b, thereby forming a closed circuit with respect to the sensor unit 190-1. In other words, the connector 180 may form an electrical connection by contacting the first terminal 190-1a and the second terminal 190-1b.

When the connector 180 is electrically connected to the first terminal 190-1a and the second terminal 190-1b, the control module 16 may generate a signal.

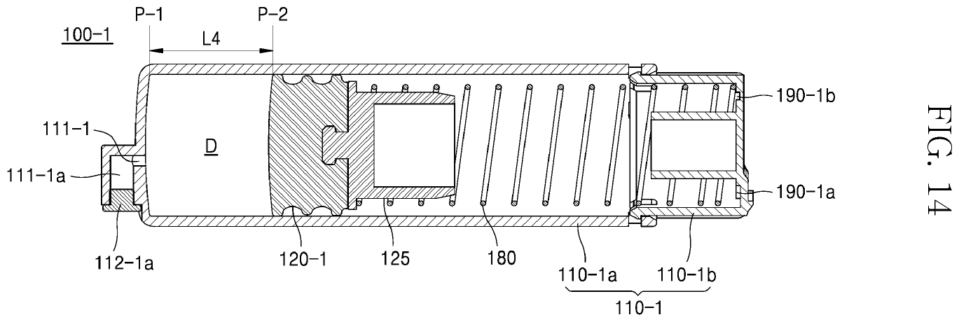

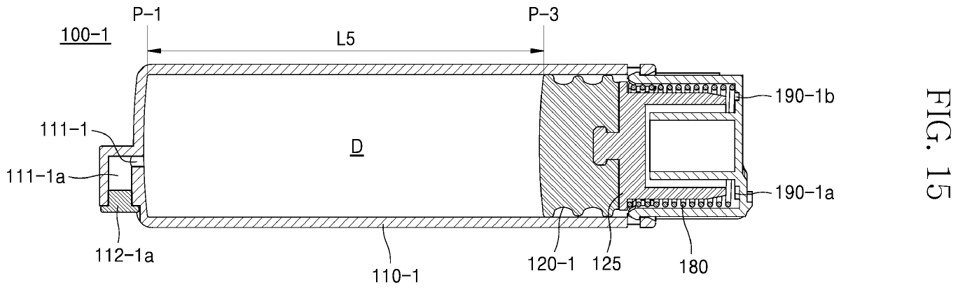

With reference to FIGS. 13 to 15, the operation of the reservoir assembly 100-1 may be described. FIG. 13 shows the state before the medical liquid D flows into the reservoir 110-1, FIG. 14 shows the state in which the medical liquid D is flowing into the reservoir 110-1, and FIG. 15 shows the state in which all the medical liquid D flowed into the reservoir 110-1.

As shown in the drawings, in the inner space of the reservoir 110-1 formed as the reservoir case 110-1a and the reservoir cap 110-1b are coupled to each other, the plunger 120-1, the plunger holder 125, the connector 180, and the sensor unit 190-1 may be accommodated. the opening 111-1 may be formed at the front side of the reservoir 110-1, and the sensor unit 190-1 including the first terminal 190-1a and the second terminal 190-1b may be formed at the rear side of the reservoir 110-1.

The plunger 120-1 may be inserted into the inner space of the reservoir 110-1, such that the front portion of the plunger 120-1 faces the opening 111-1. A fixed plunger holder coupled to the connector 180 may be coupled to the rear side of the plunger 120-1.

<The Process in which the Medical Liquid D Flows into the Reservoir 110-1>

The medical liquid D may be stored in the reservoir 110-1 in the order shown in FIGS. 13 to 15. While the medical liquid D is being stored in the reservoir 110-1, the plunger 120-1 may move from the front side to the rear side, preferably from the first position P-1 to a third position P-3.

Referring to FIG. 13, in the initial state before the medical liquid D flows into the reservoir 110-1, the plunger 120-1 may be positioned at the first position P-1 at which the front side of the plunger 120-1 contacts the front side of the reservoir 110-1 having formed therein the opening 111-1. At this time, a storage space may not be formed in the internal space of the reservoir 110-1.

The length of the connector 180 may extend to a first length L1 in the longitudinal direction of the reservoir 110-1. The first length L1 is shorter than the length (second length L2) from the front side of the connector 180 to the first terminal 190-1a and the second terminal 190-1b, and the rear side of the connector 180 may be spaced apart from the first terminal 190-1a and the second terminal 190-1b by a third length L3.

While the plunger 120-1 is moving from the first position P-1 to the second position P-2, the connector 180 does not contact the first terminal 190-1a and the second terminal 190-1b, and thus no electricity may be conducted between the first terminal 190-1a and the second terminal 190-1b.

Referring to FIG. 14, the medical liquid D flowing in through the inlet end may flow into the reservoir 110-1 through the opening 111-1 along the flow path 111-1a. As the medical liquid D flows into the reservoir 110-1, the plunger 120-1 moves backward inside the reservoir 110-1 to form a storage space, and the storage space may gradually become larger.

When the plunger 120-1 is positioned at the second position P-2 by moving a fourth length L4 from the first position P-1, the rear side of the connector 180 may contact the first terminal 190-1a and the second terminal 190-1b. At this time, the fourth length LA may be equal to the third length L3. When the rear side of the connector 180 contacts the first terminal 190-1a and the second terminal 190-1b, the connector 180 may short and electrically connect the first terminal 190-1a and the second terminal 190-1b from and to each other. When the first terminal 190-1a and the second terminal 190-1b are electrically connected to each other through the connector 180, the controller 30 may recognize a specific event of the reservoir assembly 100-1.

For example, when the connector 180 comes into contact with the first terminal 190-1a and the second terminal 190-1b, the controller 30 may recognize that the medical liquid D is stored in the reservoir 110-1 at a first reference amount (e.g., 10%, 20%, 30%, etc.). The first reference amount may vary depending on the third length L3.

When it is recognized that the medical liquid D is stored in the reservoir 110-1 at the set first reference amount, the controller 30 may wake up the medical liquid infusion apparatus 10 in a first mode. In other words, the controller 30 may check that a certain amount of a medical liquid D is stored in the reservoir 110-1 and start partial driving to pre-heat the medical liquid infusion apparatus 10. Also, the user may be notified in advance through the user terminal that a pre-set first reference amount of the medical liquid D is stored in the reservoir 110-1, and thus the user may use the medical liquid infusion apparatus 10.

Referring to FIG. 15, as the medical liquid D flows into the reservoir 110-1, the plunger 120-1 moves to the rear side of the reservoir 110-1 and, due to a reason like that the connector 180 may no longer be compressed or the plunger holder 125 contacts the rear side of the reservoir 110-1, may stop at the third position P-3 by moving a fifth length L5 from the first position P-1. At this time, the storage space may be maximized. Also, the state in which the rear side of the connector 180 is in contact with the first terminal 190-1a and the second terminal 190-1b may be maintained, thereby electrically interconnecting the first terminal 190-1a and the second terminal 190-1b.

Afterwards, the controller 30 may infuse the medical liquid D into a patient in a second mode.

<The Process in which the Medical Liquid D is Discharged from the Reservoir 110-1 to a Needle>

The medical liquid D may be discharged from the reservoir 110-1 in the order shown in FIGS. 15 to 13.

When the discharge of the medical liquid D begins, the medical liquid D stored in the reservoir 110-1 flows through the opening 111-1 and the flow path 111-1a and may then be discharged to the needle assembly 400 through a conduit (not shown) connected to the outlet end 113-1. While the medical liquid D is being discharged from the reservoir 110-1, the plunger 120-1 may move between the sensor unit 190-1 and the opening 111-1 (preferably from the third position P-3 to the first position P-1).

In the process of discharging the medical liquid D to the needle, as the plunger 120-1 passes through the second position P-2, connection of the connector 180 to the first terminal 190-1a and the second terminal 190-1b may be released.

According to an embodiment, the controller 30 may activate a third mode when the electrical connection between the first terminal 190-1a and the second terminal 190-1b is broken.

When the connector 180 is separated after maintaining contact with the first terminal 190-1a and the second terminal 190-1b, the electric connection between the first terminal 190-1a and the second terminal 190-1b may be broken. When the electrical connection between the first terminal 190-1a and the second terminal 190-1b is broken, the controller 30 may recognize a specific event of the reservoir assembly 100-1.

In the third mode, the controller 30 may transmit an alarm signal indicating that a stored amount of a medical liquid D corresponds to a second reference amount to the user through the user terminal 20, the controller 30, and/or the alarm unit 800 (not shown).

The second reference amount may be defined as an amount of the medical liquid D set by the controller 30 at the time of activation of the third mode. The controller 30 may inform the user that the amount of the medical liquid D remaining in the reservoir 110-1 is a pre-set second reference amount, and thus the user may prepare to replace the medical liquid infusion apparatus 10.

According to an embodiment, the first reference amount may be set to be the same storage amount of the medical liquid D as the second reference amount. In other words, the second reference amount may be set to be equal to the amount of medical liquid D actually stored in the reservoir 110-1. As the plunger 120-1 moves from the first position P-1 to the third position P-3 or from the third position P-3 to the first position P-1, a position at which the connector 180 and terminals 190-1a and 190-1b of the sensor unit 190-1 contact each other is identical to a position at which the connector 180 and the terminals 190-1a and 190-1b of the sensor unit 190-1 are separated from each other, and thus the first reference amount and the second reference amount may be equal to each other.

According to another embodiment, the first reference amount may be set to be a greater storage amount of the medical liquid D than the second reference amount. The first reference amount is a reference value set for operating in the first mode, and may be set to be substantially equal to an amount of the medical liquid D stored in the reservoir 110-1. However, the second reference amount is an amount of the medical liquid D recognized by the controller at a time point at which the third mode starts (connection between the connector 180 and at least one of the first terminal 190-1a and the second terminal 190-1b is released) and may be set to be smaller than the actual amount of the medical liquid D remaining in the reservoir 110-1.

In other words, when the plunger 120-1 passes the second position P-2, the actual amount of the medical liquid D remaining in the reservoir 110-1 is greater than the second reference amount recognized by the controller 30. Therefore, even when the controller 30 recognizes the risk due to exhaustion of the medical liquid D, there may be the medical liquid D of a surplus amount of the corresponding to a difference between the actual remaining amount of the medical liquid D corresponding to the second position P-2 and the second reference amount in the reservoir 110-1. Therefore, even when the medical liquid infusion apparatus 10 generates a signal that the medical liquid D is completely exhausted, the user may still use the surplus amount of the medical liquid D, thereby preventing a sudden discontinuation of the medical liquid D or an accident. As a result, the safety of the medical liquid infusion apparatus 10 may be improved.

Since the remaining amount of the medical liquid D is important in the third mode, the controller 30 may calculate the infusion amount of the medical liquid D and the amount of the medical liquid D remaining in the reservoir 110-1 very precisely in the third mode. In the third mode, based on data obtained from an encoder, etc., the controller 30 may accurately measure the rotation angle of the driving unit, which will be described later, and the moving distance of the plunger 120-1, and precisely calculate the discharge amount of the medical liquid D and the amount of the medical liquid D remaining in the reservoir 110-1. The remaining amount of the medical liquid D precisely calculated in the third mode may be transmitted to the user in real time, and thus the user may recognize the risk.

According to an embodiment, the medical liquid infusion apparatus 10 may accurately count the amount of the medical liquid D remaining in the reservoir 110-1 only in the third mode. In the second mode, since the amount of the medical liquid D stored in the reservoir 110-1 exceeds a pre-set range (i.e., the second reference amount), the amount of the medical liquid D stored in the reservoir 110-1 is not precisely measured. However, in the third mode, the amount of the medical liquid D stored in the reservoir 110-1 may be measured precisely. Since the amount of medical liquid D stored in the medical liquid infusion apparatus 10 is accurately measured only at the level at which an alarm is needed, the control load of the medical liquid infusion apparatus 10 may be reduced.

Therefore, the above-stated medical liquid infusion apparatus and the reservoir assembly 100-1 may measure the infusion amount of the medical liquid D stored in the reservoir 110-1. The amount of the medical liquid D stored in the reservoir 110-1 may be measured by the controller 30 through the sensor unit 190-1, and the operation of the medical liquid infusion apparatus 10 may be set based on the measured amount. When the plunger 120-1 moves linearly inside the reservoir 110-1, the connector 180 connected to the plunger 120-1 may also move together and the connector 180 and the sensor unit 190-1 may contact each other or be separated from each other. During the process, the amount of the medical liquid D stored in the reservoir 110-1 may be sensed.

The medical liquid infusion apparatus and reservoir assembly 100-1 may accurately measure the amount of medical liquid D stored in the reservoir 110-1 while reducing the overall volume by using the compressible connector 180. Also, by placing the connector 180 and the sensor unit 190-1 in an internal space provided by the reservoir 110-1, the apparatus may be simplified and the size thereof may be reduced.

The medical liquid infusion apparatus and reservoir assembly 100-1 may be pre-heated when the reservoir 110-1 is filled with a certain amount of the medical liquid D, thereby improving driving efficiency. When the sensor unit 190-1 senses that the amount of medical liquid D infused into the reservoir 110-1 is equal to or greater than the first reference amount, the medical liquid infusion apparatus 10 prepares to drive some of parts thereof in the first mode, and, when the medical liquid infusion apparatus 10 is attached to a user, the medical liquid infusion apparatus 10 may immediately infuse the medical liquid D.

The medical liquid infusion apparatus and the reservoir assembly 100-1 may sense and notify the user when the amount of the medical liquid D stored in the reservoir 110-1 is dropped to or below a predetermined amount. When the sensor unit 190-1 senses that the amount of medical liquid stored in the reservoir 110-1 is dropped to or below the second reference amount, the medical liquid infusion apparatus 10 may precisely count the amount of the medical liquid remaining in the reservoir 110-1 and provide information regarding the amount of the medical liquid remaining in the reservoir 110-1 to a user.

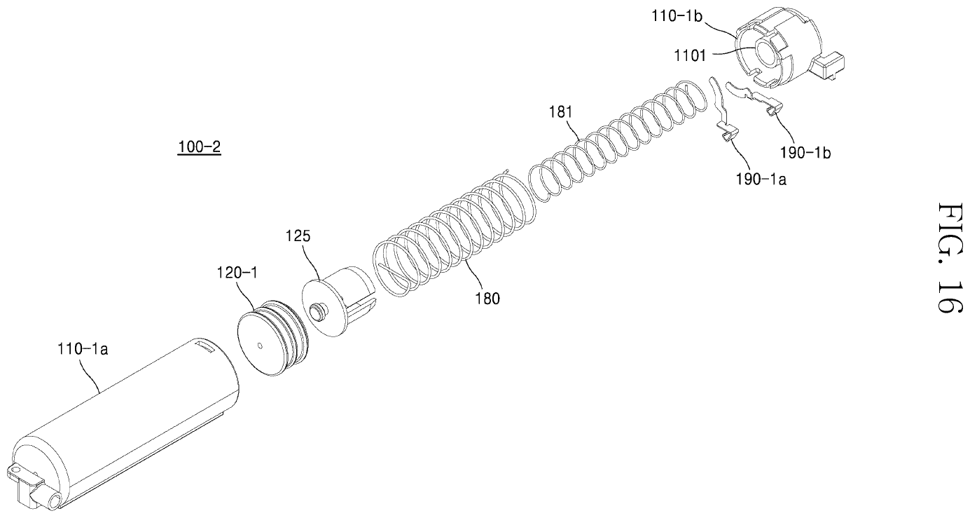

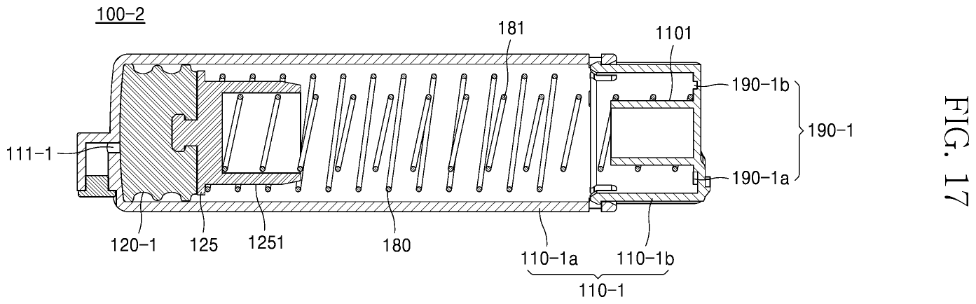

FIG. 16 is an exploded perspective view of a reservoir assembly 100-2 further including an auxiliary pressing member 181, and FIG. 17 is a cross-sectional view of FIG. 16.

Referring to FIGS. 16 to 17, the reservoir assembly 100-2 further includes the auxiliary pressing member 181, and a pressing member fixing portion 1101 by which the auxiliary pressing member 181 may be fixed may be further formed at the rear side of the reservoir 110-1 or at the reservoir cap 110-1b. According to an embodiment, the pressing member fixing portion 1101 may be formed in a cylindrical shape extending a predetermined length from the rear side of the reservoir 110-1 to the front side of the reservoir 110-1. The rear side of the auxiliary pressing member 181 may surround the outer peripheral surface of the pressing member fixing portion 1101 and may secure fixing force.

In the internal space provided by the reservoir 110-1, the auxiliary pressing member 181 may be disposed at a position from the rear side of the plunger 120-1 to the rear side of the reservoir 110-1 and press the plunger 120-1 forward. According to an embodiment, the auxiliary pressing member 181 may be disposed to press the plunger 120-1 forward while the front side of the auxiliary pressing member 181 is connected to the plunger 120-1 and the rear side of the auxiliary pressing member 181 is fixed to the pressing member fixing portion 1101. At this time, the auxiliary pressing member 181 may not contact the sensor unit 190-1 disposed at the rear side of the reservoir 110-1.

The auxiliary pressing member 181 is not limited to a specific shape and may have various shapes. For example, the auxiliary pressing member 181 may be a coiled spring or a spiral spring. However, for convenience of explanation, descriptions below will focus on an embodiment in which the auxiliary pressing member 181 is provided as a coiled spring as shown in the drawings. Also, the auxiliary pressing member 181 may not be conductive to not to form an electrical connection with the sensor unit 190-1.

The auxiliary pressing member 181 may extend rearward while the front side thereof is connected to the plunger holder 125, but may not cause interference with the connector 180. According to an embodiment, the auxiliary pressing member 181 may be formed as a coil spring having a smaller diameter than the connector 180, and thus the auxiliary pressing member and the connector 180 may each be coupled to the plunger holder 125. At this time, the auxiliary pressing member 181 and the connector 180 may be coupled to the plunger holder 125, such that the auxiliary pressing member 181 is inserted into the inside of the connector fixing portion 1251, and the connector 180 surrounds the outer peripheral surface of the connector fixing portion 1251. The auxiliary pressing member 181 is disposed inside the connector 180, and thus the auxiliary pressing member 181 and the connector 180 may not interfere with each other even while the auxiliary pressing member 181 and the connector 180 are being compressed.

Referring to the drawings, a configuration in which the auxiliary pressing member has a smaller diameter than the connector 180 and is disposed inside the connector 180 has been described. However, in the configuration in which the connector 180 may contact the sensor unit 190-1, the arrangements of the auxiliary pressing member 181 and the connector 180 may be interchanged.

Referring to FIGS. 13 to 15, while a medical liquid is being stored in the reservoir 110-1, the plunger 120-1 may move from the front side to the rear side, preferably from the first position P-1 to the third position P-3. Also, referring to FIGS. 13 to 15 in the reversed order, while a medical liquid is being discharged from the reservoir 110-1, the plunger 120-1 may move from the rear side to the front side, preferably from the third position P-3 to the first position P-1. In other words, the plunger 120-1 may move due to the medical liquid flowing into and out of the reservoir 110-1 through the opening 111-1.

However, while the medical liquid is flowing out of the reservoir 110-1, the plunger 120-1 may not move as much as a distance corresponding to the amount of the medical liquid flowing out of the reservoir 110-1 due to the friction formed on the outer peripheral surface of the plunger 120-1 and the inner peripheral surface of the reservoir 110-1. When the movement of the plunger 120-1 corresponding to the medical liquid flowing out of the reservoir 110-1 is not smooth, negative pressure may be formed in the storage space and an external gas may flow in, and thus bubbles may be formed in the medical liquid stored in the storage space.

The auxiliary pressing member 181 may press the plunger 120-1 forward, thereby preventing formation of bubbles in the medical liquid stored in the storage space while the medical liquid is flowing out.

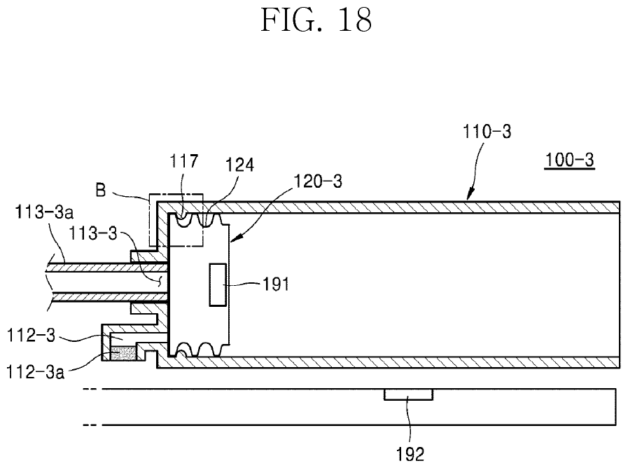

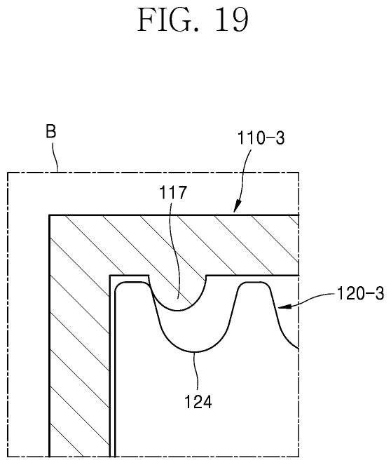

FIG. 18 is a side cross-sectional view of a reservoir assembly 100-3 according to another embodiment of the present disclosure, and FIG. 19 is an enlarged view of a portion B of FIG. 18.

Referring to FIGS. 18 and 19, the reservoir assembly 100-3 may store a medical liquid in at least a portion of an internal space thereof and discharge a set amount of the medical liquid to the needle assembly 400 by using the driving force generated by operations of a driving unit and a driving module, which will be described later.

The reservoir assembly 100-3 may include a reservoir 110-3, a plunger 120-3, and a sensor unit 190-3.

The reservoir 110-3 forms the outer portion of the reservoir assembly 100-3 and may provide an internal space in which the plunger 120-3 may be accommodated. A portion of the internal space of the reservoir 110-3 is formed as a storage space for a medical liquid, and the storage space may be defined by the reservoir 110-3 and the plunger 120-3.

According to an embodiment, the reservoir 110-3 may have a cylindrical shape. The reservoir 110-3 may be in the form of a cylinder with a closed front portion. Also, the rear portion of the reservoir 110-3 may be opened to form an open internal space. Alternatively, the rear portion of the reservoir 110-3 may be closed to form a closed internal space.

The reservoir 110-3 may have an inlet end 112-3 and an outlet end 113-3 formed at the front side through which a medical liquid may flow. The medical liquid may flow into the internal space of the reservoir 110-3 through the inlet end 112-3 to form a storage space, and the medical liquid stored in the storage space may be discharged through the outlet end 113-3. Although the drawings illustrate the reservoir 110-3 in which the inlet end 112-3 and the outlet end 113-3 are formed at the front side, one opening may be formed at the front side of the reservoir 110-3, and the opening may be configured to communicate with the inlet end 112-3 through which the medical liquid is infused and the outlet end 113-3 through which the medical liquid is discharged.

A packing member 112-3a is provided at the inlet end 112-3 to prevent the medical liquid infused into the reservoir 110-3 through the inlet end 112-3 from leaking out. Also, an infusion needle LF (not shown) may be inserted through the packing member 112-3a and the medical liquid may be infused.

The packing member 112-3a may have reclosable properties. For example, even when the infusion needle LF (not shown) penetrates through the packing member 112-3a and then is removed to fill the reservoir 110-3 with a medical liquid, the packing member 112-3a may prevent the medical liquid from leaking out through a portion of the packing member 112-3a penetrated by the infusion needle LF (not shown).

The packing member 112-3a may include a material containing polypropylene, thermoplastic elastomer, vegetable oil, etc.

Infused medical liquid may push the plunger 120-3 backward and flow into the internal space of the reservoir 110-3, thereby forming a storage space.

A conduit 113-3a may be connected to the outlet end 113-3, and the reservoir 110-3 may be connected to a driving unit to be described later through the conduit 113-3a. The medical liquid stored in the storage space may flow to the needle assembly 400 through the outlet end 113-3 and the conduit 113-3a connected to the outlet end 113-3.

Due to the flow of the medical liquid inside the reservoir 110-3, the plunger 120-3 may move in at least one of the forward direction and the backward direction in the internal space of the reservoir 110-3. In the internal space of the reservoir 110-3, a storage space may be defined by the reservoir 110-3 and the front side of the plunger 120-3. The front side of the plunger 120-3 forms a storage space for a medical liquid together with the reservoir 110-3, and, when the medical liquid is stored in the reservoir 110-3, the front side of the plunger 120-3 may contact the medical liquid.

The outer peripheral surface of the plunger 120-3 is in close contact with the inner peripheral surface of the reservoir 110-3 to prevent the medical liquid stored in the storage space from leaking out. For example, the plunger 120-3 may include an elastic material such as rubber or silicon and may be in close contact with the inner peripheral surface of the reservoir 110-3.

At least one groove 124 may be formed on the outer peripheral surface of the plunger 120-3. The groove 124 may be formed in the circumferential direction on the outer peripheral surface of the plunger 120-3. When a plurality of grooves 124 are formed on the outer peripheral surface of the plunger 120-3, the plurality of grooves may be arranged in the longitudinal direction of the plunger 120-3.

The reservoir 110-3 may have a protrusion 117 formed on the inner peripheral surface of the reservoir 110-3. The plunger 120-3 is maintained at an initial position set by the protrusion 117 and may move to the rear side of the reservoir 110-3 as the medical liquid flows into the reservoir 110-3. In other words, the plunger 120-3 maintains its initial position by the protrusion 117, but may move rearward by the medical liquid flowing into the reservoir 110-3.

The protrusion 117 may be disposed inside the reservoir 110-3 and protrude from the inner peripheral surface of the reservoir 110-3. For example, the protrusion 117 may be formed to protrude in an annular shape from the inner peripheral surface of the reservoir 110-3. The protrusion 117 may be disposed adjacent to at least one of the inlet end 112-3 and the outlet end 113-3.

Before the medical liquid is infused into the reservoir 110-3, the plunger 120-3 may be positioned at the initial position in front of the reservoir 110-3 while the front side of the plunger 120-3 is in close contact with the reservoir 110-3. When the medical liquid is infused into the reservoir 110-3, the plunger 120-3 may move from the front side to the rear side due to the infused medical liquid. Also, when all of the medical liquid is infused, the plunger 120-3 may stop moving and be positioned at a final position behind the reservoir 110-3.

When, before the medical liquid is infused into the reservoir 110-3, a pressure change occurs inside the medical liquid infusion apparatus due to injection of a sterilizing gas or vibration or shock is applied to the medical liquid infusion apparatus, the plunger 120-3 may be moved from its initial position, and thus the front side of the plunger 120-3 may be separated from the reservoir 110-3 and a gas may flow into the reservoir 110-3. At this time, when the medical liquid is infused into the reservoir 110-3, the amount of the medical liquid stored in the reservoir 110-3 becomes less than a preset amount, and thus a set amount of the medical liquid may not be provided to a user. Also, bubbles may be formed in the stored medical liquid, which may cause safety problems.

When the plunger 120-3 is at its initial position, the protrusion 117 may be inserted into the groove 124 to restrict the plunger 120-3 from moving rearward. The protrusion maintains the position of the plunger 120-3 to prevent the front side of the plunger 120-3 from being separated from the reservoir 110-3 until the medical liquid is infused into a user, thereby maintaining the amount of the medical liquid that may be stored in a storage space.

Referring to FIG. 19, the protrusion 117 protruding from the inner peripheral surface of the reservoir 110-3 may have the curved front side and the curved rear side. For example, the protrusion 117 may have a side cross-section having a partially circular or elliptical shape. When the plurality of grooves 124 are formed on the outer peripheral surface of the plunger 120-3, the protrusion 117 may be formed to be inserted into the foremost groove 124.

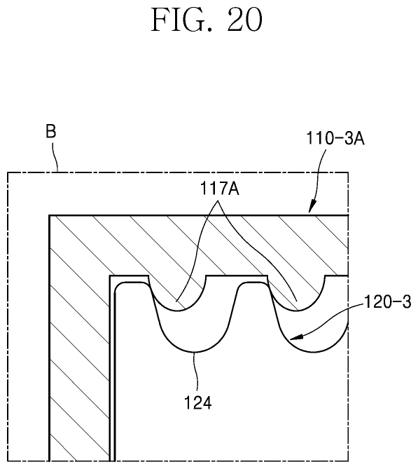

FIG. 20 is a diagram illustrating a protrusion according to an embodiment different from that of FIG. 19.

Referring to FIG. 20, a plurality of protrusions 117A may be formed to protrude from the front side of a reservoir 110-3A. The plurality of protrusions 117A may be respectively inserted into the plurality of grooves 124 formed on the outer peripheral surface of the plunger 120-3 to maintain the initial position of the plunger 120-3. The plunger 120-3 may more stably maintain its initial position by the plurality of protrusions 117A and the plurality of grooves 124.

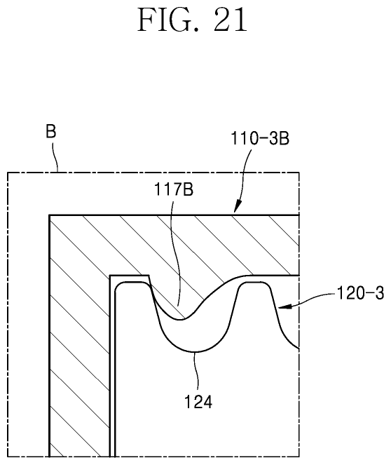

FIG. 21 is a diagram illustrating a protrusion according to an embodiment different from that of FIG. 19.

Referring to FIG. 21, the front side of a protrusion 117B may be formed with a greater curvature than the rear side of the protrusion 117B. Since the front side of the protrusion 117B has a greater curvature than the rear side of the protrusion 117B, the protrusion 117B may allow the plunger 120-3 to move forward relatively easily while restricting the plunger 120-3 from moving rearward. Therefore, the protrusion 117B may prevent a gas from flowing into a reservoir 110-3B before a medical liquid is infused and may not prevent the medical liquid stored in the reservoir 110-3B from being completely discharged.

Referring back to FIG. 18, the sensor unit 190-3 is a component for detecting the infusion of a medical liquid into the reservoir 110-3 and may transmit a signal generated by the movement of the plunger 120-3 within the reservoir 110-3. The sensor unit 190-3 may be connected to a FPCB, and thus the sensor unit 190-3 may be electrically connected to a controller. The sensor unit 190-3 may be provided as a variety of sensors capable of sensing the position of a component, such as an optical sensor or a contact sensor.

According to an embodiment, the sensor unit 190-3 may be configured as a magnetic sensor including a magnetic member 191 and a magnetic sensing member 192. The magnetic member 191 may be installed at the plunger 120-3 and move together with the plunger 120-3. The magnetic sensing member 192 may be installed at a predetermined position outside the reservoir 110-3.

The positions of the magnetic member 191 and the magnetic sensing member 192 in the sensor unit 190-3 may be reversed. In other words, the magnetic sensing member 192 may be installed at the plunger 120-3, and the magnetic member 191 may be installed at a predetermined position outside the reservoir 110-3. However, hereinafter, for convenience of explanation, descriptions will be given based on an embodiment in which the magnetic member is installed at the plunger 120-3 and the magnetic sensing member 192 is installed at a predetermined position outside the reservoir 110-3 as shown in the drawings.

The sensor unit 190-3 may determine the position of the plunger 120-3 by generating a signal by the magnetic force formed between the magnetic member 191 and the magnetic sensing member 192.

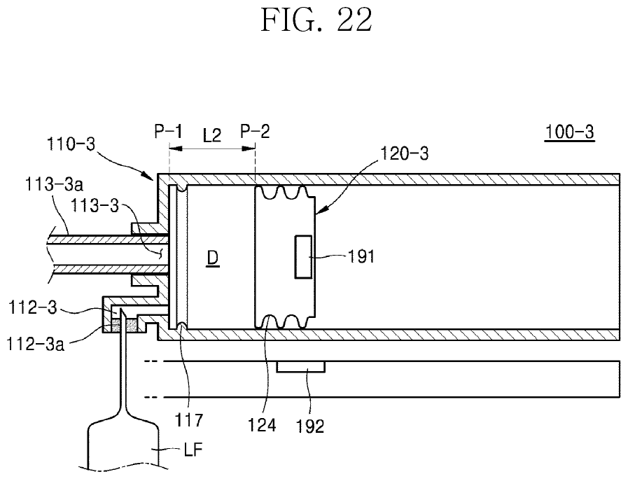

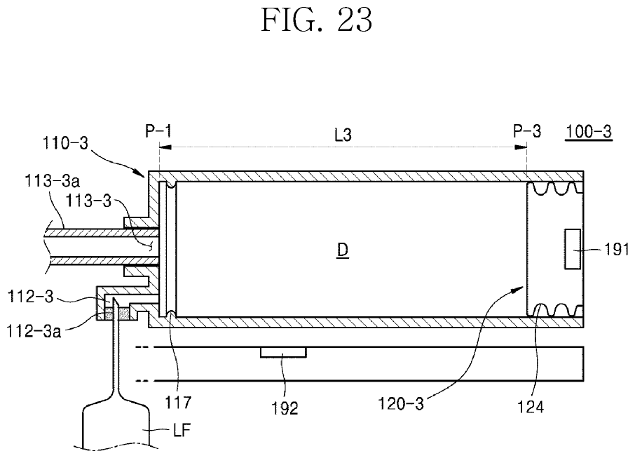

FIG. 22 is a diagram showing a state in which a predetermined amount of a medical liquid is infused in FIG. 18, and FIG. 23 is a diagram showing a state in which the maximum amount of the medical liquid is infused in FIG. 18.

With reference to FIGS. 18, 22, and 23, the operation of the reservoir assembly 100-3 may be described. FIG. 18 shows the state before the medical liquid D flows into the reservoir 110-3, FIG. 22 shows the state in which a predetermined amount of the medical liquid D is flowing into the reservoir 110-3, and FIG. 15 shows the state in which a set amount of the medical liquid D flowed into the reservoir 110-3.

As shown in the drawings, the inlet end 112-3 and the outlet end 113-3 may be formed at the front side of the reservoir 110-3. The plunger 120-3 having installed thereon the magnetic member 191 is inserted into the inner space of the reservoir 110-3, and the front side of the plunger 120-3 may face the inlet end 112-3 and the outlet end 113-3.

In FIG. 18, before the medical liquid is infused, the plunger 120-3 may be positioned at an initial position where the front surface of the plunger 120-3 may be in close contact with the reservoir 110-3. At this time, the protrusion 117 may be inserted into the groove to allow the plunger 120-3 to maintain its initial position. For example, the protrusion 117 may protrude in an annular shape from the inner peripheral surface of the reservoir 110-3 and may be inserted into the groove 124 formed annularly on the outer peripheral surface of the plunger 120-3. As the plunger 120-3 maintains its initial position by the protrusion 117, it is possible to prevent a gas from flowing into the reservoir 110-3 due to external factors.

<The Process in which the Medical Liquid D Flows into the Reservoir 110-3>

The medical liquid D may be stored in the reservoir 110-1 in the order shown in FIGS. 18, 22, and 23. While the medical liquid D is being stored in the reservoir 110-3, the plunger 120-3 may move from the front side to the rear side, preferably from the first position P-1 to a third position P-3.

Referring to FIG. 18, in the initial state before the medical liquid D flows into the reservoir 110-3, the plunger 120-3 may be positioned at the first position P-1 at which the front side of the plunger 120-3 contacts the front side of the reservoir 110-3 having formed therein the inlet end 112-3 and the outlet end 113-3. At this time, a storage space may not be formed in the internal space of the reservoir 110-3. The inlet end 112-3 and the outlet end 113-3 formed in the front side of the reservoir 110-3 are blocked by the front surface of the plunger 120-3, and thus introduction of a gas into the internal space may be prevented.

Referring to FIG. 22, as the medical liquid D is infused, the plunger 120-3 may move rearward from the first position P-1 to form a storage space. While the plunger 120-3 is moving the second length L2, the magnetic force formed between the magnetic member 191 and the magnetic sensing member 192 may gradually increase. While the plunger 120-3 is positioned at the second position P-2, the magnetic force formed between the magnetic member 191 and the magnetic sensing member 192 may be maximized. When the plunger 120-3 moves rearward from the second position P-2, the magnetic force formed between the magnetic member 191 and the magnetic sensing member 192 may gradually decrease.

The controller 30 may detect a change in the magnetic force due to the movement of the plunger 120-3 and generate a specific event of the reservoir assembly 100. For example, when the magnetic force formed between the magnetic member 191 and the magnetic sensing member 192 is maximized (i.e., when the plunger 120-3 is at the second position P-2), the controller 30 may recognize that the medical liquid D is stored in the reservoir 110-1 at a first reference amount (e.g., 10%, 20%, 30%, etc.). The first reference amount may vary depending on the position at which the magnetic sensing member 192 is installed.

When it is recognized that the medical liquid D is stored in the reservoir 110-3 at the set first reference amount, the controller 30 may wake up a medical liquid infusion apparatus in the first mode. In other words, the controller 30 may check that a certain amount of a medical liquid D is stored in the reservoir 110-3 and start partial driving to pre-heat the medical liquid infusion apparatus. Also, the user may be notified in advance through the user terminal 20 that a pre-set first reference amount of the medical liquid D is stored in the reservoir 110-3, and thus the user may use the medical liquid infusion apparatus.

Referring to FIG. 23, the plunger 120-3 may move to the rear side of the reservoir 110-3 due to introduction of the medical liquid D and, when a set amount of the medical liquid D flows into the reservoir 110-3, the plunger 120-3 may stop at the third position P-3 that is the third length L3 apart from the first position P-1. At this time, the storage space may be maximized.

Afterwards, the controller 30 may infuse the medical liquid D into a patient in a second mode.

<The Process in which the Medical Liquid D is Discharged from the Reservoir 110-3 to a Needle>

The medical liquid D may be discharged from the reservoir 110-3 in an order opposite to the order shown in FIGS. 18, 22, and 23.

When the discharge of the medical liquid D begins, the medical liquid D stored in the reservoir 110-3 may be discharged to the needle assembly 400 through the conduit 113-3a connected to the outlet end 113-3. While the medical liquid D is being discharged from the reservoir 110-3, the plunger 120-3 may move from the third position P-3 to the first position P-1.

In the process of discharging the medical liquid D from the reservoir 110-3, as the plunger 120-3 passes through the second position P-2, the magnetic force formed between the magnetic member 191 and the magnetic sensing member 192 may be maximized. At this time, the controller 30 may recognize the event as a specific event of the reservoir assembly 100-3 and activate the third mode.

In the third mode, the controller 30 may transmit an alarm signal indicating that the amount of the medical liquid D remaining in the reservoir 110-3 corresponds to a second reference amount to the user through the user terminal 20, the controller 30, and/or an alarm unit (not shown).

The second reference amount may be defined as an amount of the medical liquid D set by the controller 30 at the time of activation of the third mode. The controller 30 may inform the user that the amount of the medical liquid D remaining in the reservoir 110-3 is a pre-set second reference amount, and thus the user may prepare to replace the medical liquid infusion apparatus.

According to an embodiment, the first reference amount may be set to be the same storage amount of the medical liquid D as the second reference amount. In other words, the second reference amount may be set to be equal to the amount of medical liquid D actually stored in the reservoir 110-3. The maximum value of the magnetic force formed between the magnetic member 191 and the magnetic sensing member 192 as the plunger 120-3 moves from the first position P-1 to the third position P-3 is identical to the maximum value of the magnetic force formed between the magnetic member 191 and the magnetic sensing member 192 as the plunger 120-3 moves from the third position P-3 to the first position P-1, the first reference amount and the second reference amount may be set to be equal to each other.

According to another embodiment, the first reference amount may be set to be a greater storage amount of the medical liquid D than the second reference amount. The first reference amount is a reference value set for operating in the first mode, and may be set to be substantially equal to an amount of the medical liquid D stored in the reservoir 110-3. However, the second reference amount is the amount of medical liquid D recognized by the controller 30 when the third mode starts (the plunger 120-3 passes the second position P-2) and may be set to be smaller than the actual amount of the medical liquid D remaining in the reservoir 110-3.

In other words, when the plunger 120-3 passes the second position P-2, the actual amount of the medical liquid D remaining in the reservoir 110-3 is greater than the second reference amount recognized by the controller 30. Therefore, even when the controller 30 recognizes the risk due to exhaustion of the medical liquid D, there may be the medical liquid D of a surplus amount of the corresponding to a difference between the actual remaining amount of the medical liquid D corresponding to the second position P-2 and the second reference amount in the reservoir 110-3. Therefore, even when the medical liquid infusion apparatus generates a signal that the medical liquid D is completely exhausted, the user may still use the surplus amount of the medical liquid D, thereby preventing a sudden discontinuation of the medical liquid D or an accident. As a result, the safety of the medical liquid infusion apparatus may be improved.

Since the remaining amount of the medical liquid D is important in the third mode, the controller 30 may calculate the infusion amount of the medical liquid D and the amount of the medical liquid D remaining in the reservoir 110-3 very precisely in the third mode. In the third mode, based on data obtained from an encoder, etc., the controller 30 may accurately measure the rotation angle of the driving unit, which will be described later, and the moving distance of the plunger 120-3, and precisely calculate the discharge amount of the medical liquid D and the amount of the medical liquid D remaining in the reservoir 110-3. The remaining amount of the medical liquid D precisely calculated in the third mode may be transmitted to the user in real time, and thus the user may recognize the risk.

According to an embodiment, the medical liquid infusion apparatus may accurately count the amount of the medical liquid D remaining in the reservoir 110-3 only in the third mode. In the second mode, since the amount of the medical liquid D stored in the reservoir 110-3 exceeds a pre-set range (i.e., the second reference amount), the amount of the medical liquid D stored in the reservoir 110-3 is not precisely measured. However, in the third mode, the amount of the medical liquid D stored in the reservoir 110-3 may be measured precisely. Since the amount of medical liquid D stored in the medical liquid infusion apparatus is accurately measured only at the level at which an alarm is needed, the control load of the medical liquid infusion apparatus may be reduced.

The above-stated medical liquid infusion apparatus and the reservoir assembly 100-3 may measure the infusion amount of the medical liquid D stored in the reservoir 110-3. The amount of the medical liquid D stored in the reservoir 110-3 may be measured by the controller 30 through the sensor unit 190-3, and the operation of the medical liquid infusion apparatus may be set based on the measured amount.

The medical liquid infusion apparatus and the reservoir assembly 100-3 may restrict the movement of the plunger 120-3 to prevent the plunger 120-3, which moves according to the flow of a medical liquid, from moving from its initial position due to other factors, thereby providing a set amount of the medical liquid to a user.

The medical liquid infusion apparatus and reservoir assembly 100-3 may be pre-heated when the reservoir 110-3 is filled with a certain amount of the medical liquid D, thereby improving driving efficiency. When the sensor unit 190-3 senses that the amount of medical liquid D infused into the reservoir 110-3 is equal to or greater than the first reference amount, the medical liquid infusion apparatus prepares to drive some of parts thereof in the first mode, and, when the medical liquid infusion apparatus is attached to a user, the medical liquid infusion apparatus may immediately infuse the medical liquid D.

The medical liquid infusion apparatus and the reservoir assembly 100-3 may sense and notify the user when the amount of the medical liquid D stored in the reservoir 110-3 is dropped to or below a predetermined amount. When the sensor unit 190-3 senses that the amount of medical liquid stored in the reservoir 110-3 is dropped to or below the second reference amount, the medical liquid infusion apparatus 10 may precisely count the amount of the medical liquid remaining in the reservoir 110-3 and provide information regarding the amount of the medical liquid remaining in the reservoir 110-3 to a user.

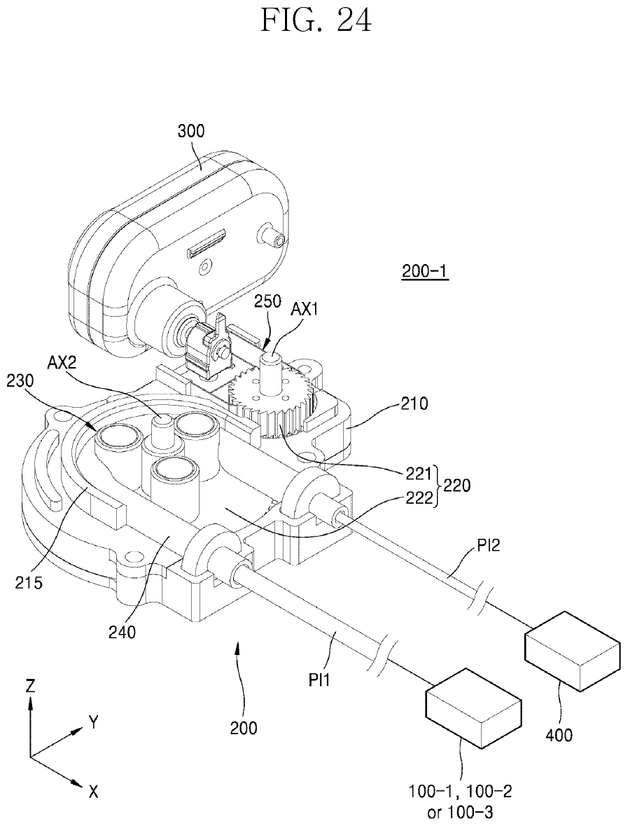

FIG. 24 is a perspective view of a driving unit 200-1 and the driving module 300, according to another embodiment of the present disclosure.

Referring to FIG. 24, the driving unit 200-1 is connected to the driving module and may be driven by the driving force generated by the driving module 300.

The driving unit 200-1 includes a base 210, a rotation unit 220, a pressing unit 230, a tube 240, and a drive piece 250, and the driving module 300 may be connected to the drive piece 250.

The base 210 supports the driving unit 200-1 and may form an outer portion of the driving unit 200-1. At least one of the rotation unit 220, the pressing unit 230, the tube 240, and the drive piece 250 may be installed on and supported by the base 210.

The base 210 may include a guide portion 215. The guide portion 215 may extend along a circumferential direction of a second rotation member 222 and support the tube 240. One portion of the guide portion 215 protrudes from one surface of the base 210, and may extend along a curve section of the tube 240. In addition, the other portion of the guide portion 215 may extend to a straight section of the tube 240.

The guide portion 215 may support the force applied by the pressing unit 230 to guide the movement of a medical liquid. When the pressing unit 230 applies the tube 240, the guide portion 215 supports the tube 240 on the opposite side of the pressing unit 230. When the pressing unit 230 applies the tube 240 at the curve section, the tube 240 may be compressed so that an internal cross-sectional area through which the medical liquid flows is zero at pressing points where the tube 240 and a pressing portion contact. At this time, when the pressing unit rotates, the medical liquid in the tube 240 also moves.

According to an embodiment, the guide portion 215 may be disposed on an outside of the curve section, and a region where the pressing unit 230 applies the tube 240 may be disposed on an inside of the curve section.

Although the drawing illustrates an embodiment in which the guide portion 215 is disposed on the outside of the tube 240 and the pressing unit 230 is disposed on the inside of the tube 240, it is not limited thereto, and in another embodiment, the guide portion may be disposed on the inside of the tube, and the pressing unit may be disposed on the outside of the tube.

The rotation unit 220 is mounted on the front side of the base 210 and may rotate by receiving the driving force from the driving module 300. The rotation unit 220 is in contact with an end of the drive piece 250, and may rotate in one direction according to the linear reciprocating motion of the drive piece 250. The rotation unit 220 may be defined as a configuration in which at least some components are rotated by receiving the driving force from the driving module 300 to rotate a pressing unit 230.

According to an embodiment, the rotation unit 220 may include a plurality of members drivably connected to each other. The rotation unit 220 may include a first rotation member 221 and the second rotation member 222.

The first rotation member 221 may contact the end of the drive piece 250 and rotate according to the linear reciprocating motion of the drive piece 250. The second rotation member 222 may be connected to the first rotation member 221 to rotate according to the rotation of the first rotation member 221. The second rotation member 222 has a plate shape and may rotate about a second axis AX2.

A discharge amount of a medical liquid is set according to the rotation angle of the second rotation member 222. In other words, the second rotation member 222 rotates and the medical liquid inside the tube 240 is discharged in a set amount according to the rotation angle of the pressing unit 230. The rotation angle and the rotation speed of the second rotation member may be set according to the number of movements of a drive shaft 310 and the number of teeth of the rotation unit 220.

The pressing unit 230 is mounted on the rotation unit 220, and may rotate together with the rotation unit 220. The pressing unit 230 may apply the tube 240 while rotating around the second axis AX2. The pressing unit 230 may apply the tube 240 when in contact with the curve section of the tube 240 such that the tube 240 is compressed at a contact point.

The pressing unit 230 may include a plurality of rollers. When the driving unit 200-1 is driven, at least one pressing portion may be disposed on the curve section. More preferably, when the pressing unit 230 rotates, at least two or more pressing portions may form the pressing points in the curve section.

As described above, at the contact point between the pressing portion of the pressing unit 230 and the tube 240, the tube is compressed by the pressing portion, so that the internal cross-sectional area of the tube 240 is formed to be zero. Since at least two or more pressing points are formed in the curve section, a set amount of a medical liquid may be discharged according to a rotation angle of the second rotation member 222.

The tube 240 is disposed adjacent to the rotation unit 220, and may include the curve section of which at least a portion extends in the circumferential direction. The tube 240 is formed of a flexible material and may be compressed by the pressing portion of the pressing unit 230.

The tube 240 may be installed between the reservoir assembly 100-1, 100-2, or 100-3 and the needle assembly 400 and may be installed to pass through the rotation unit 220. A portion of the tube 240 may extend in the circumferential direction of the second rotation member 222.

One end of the tube 240 is connected to a first conduit PI1, and thus the medical liquid in the reservoir 110-1 or 110-3 may move. The other end of the tube 240 is connected to a second conduit PI2 and may be discharged to the needle of the needle assembly 400.

The drive piece 250 may be disposed between the driving module 300 and the rotation unit 220 to transmit the driving force generated by the driving module 300 to the rotation unit 220. The drive piece 250 is connected to the drive shaft 310 so as to reciprocate linearly according to the movement of the drive shaft 310.

The medical liquid infusion apparatus 10 discharges the medical liquid D from the reservoir 110-1 or 110-3 to the needle assembly 400 by driving the driving unit 200-1. When the driving force generated by the driving module 300 is transmitted to the driving unit 200-1, the rotation unit 220 of the driving unit 200-1 rotates, and the pressing unit 230 presses a tube to move the medical liquid D.

In other words, the reservoir assembly 100-1, 100-2, or 100-3 has no additional component for moving the plunger 120-1 or 120-3. By rotating the pressing unit 230, the medical liquid (D) may be discharged from the reservoir 110-1 or 110-3 to the needle. Therefore, since no complicated mechanism for driving the plunger 120-1 or 120-3 is needed, the reservoir assembly 100-1, 100-2, or 100-3 may be configured compactly. Also, since a compact reservoir assembly 100-1, 100-2, or 100-3 is provided, the overall size of the medical liquid infusion apparatus 10 may be reduced.

Since a constant pressure is applied from the outside to the reservoir assembly 100-1, 100-2, or 100-3, the medical liquid may be discharged in a set amount. The front side of the reservoir assembly 100-1, 100-2, or 100-3 is in contact with the medical liquid, and the rear side of the plunger 120-1 or 120-3 is in communication with the outside, and thus the external atmospheric pressure is applied to the rear side of the plunger 120-1 or 120-3. Therefore, when a certain driving force is transmitted from the driving unit 200-1, a certain amount of the medical liquid D may be discharged to the needle.

Referring to FIG. 12, in the internal space of the reservoir 110-1, a portion connected to the connector 180 is connected to the outside of the reservoir 110-1. Therefore, the portion of the internal space of the reservoir 110-1 connected to the connector 180 may always maintain a constant external atmospheric pressure. The reservoir assembly 100-1 may always maintain a constant external pressure, because the front side of the plunger 120-1 is connected to the outside. Therefore, when a certain driving force is transmitted from the driving unit 200-1, a certain amount of the medical liquid D may be discharged to the needle.

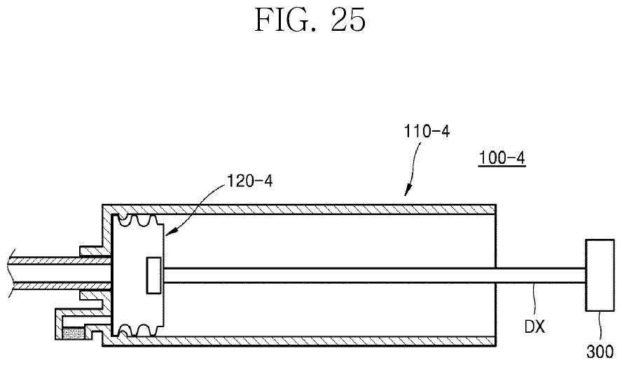

FIG. 25 is a diagram showing a reservoir assembly 100-4 according to another embodiment of the present disclosure.

Referring to FIG. 25, the reservoir assembly 100-4 is similar to the reservoir assembly 100-3 of the previous embodiment. However, since there is a difference in a driving mechanism for driving the plunger 120-4, descriptions below will focus thereon.

A plunger 120-4 is connected to a drive shaft DX, and the drive shaft DX may be selectively connected to the driving module 300 by a clutch. When the clutch is activated, the drive shaft DX may receive the driving force generated by the driving module 300. The plunger 120-4 moves rearward as a medical liquid is infused. Here, when the driving force generated by the driving module 300 is transmitted to the drive shaft DX, the plunger 120-4 may move forward and discharges the medical liquid.

The technical spirit of the present disclosure should not be construed to the above-described embodiments, and not only the claims to be described later, but also anything equivalent to or equivalently modified from these claims fall within the scope of the spirit of the present disclosure.

What is claimed is:

1. A reservoir assembly comprising:
a reservoir that provides a space therein;
a plunger that is inserted into the reservoir and moves back and forth in one direction of the reservoir to form a storage space for a medical liquid;
a rod fixed to the plunger and extending in the one direction;
a connecting member screw-coupled to the rod and movable in relation to the rod; and
a pressing member disposed to generate a pushing force between the plunger and the connecting member when the plunger and the connecting member are adjacent to each other.

2. The reservoir assembly of claim 1, wherein screw thread are formed on an outer peripheral surface of the rod, screw threads are formed on an inner peripheral surface of the connecting member, and the rod is screw-coupled to the connecting member while being inserted into the connecting member.

3. The reservoir assembly of claim 2, wherein screw threads are formed in a predetermined region of the connecting member.

4. The reservoir assembly of claim 1, wherein the pressing member is provided as a spring.

5. The reservoir assembly of claim 1, wherein the pressing member surrounds a front end of the rod and extends toward the connecting member while a front end of the pressing member is fixed to the plunger.

6. A medical liquid infusion apparatus comprising:
a reservoir assembly in which a medical liquid is stored;
a needle assembly fluidly connected to the reservoir assembly to discharge the medical liquid; and
a driving unit connected to the reservoir assembly and configured to move the medical liquid from the reservoir to the needle assembly when driven,
wherein the reservoir assembly comprises:
a reservoir that provides a space therein;
a plunger that is inserted into the reservoir and moves back and forth in one direction of the reservoir to form a storage space for a medical liquid;
a rod fixed to the plunger and extending in a direction away from the storage space;
a connecting member screw-coupled to the rod and movable in relation to the rod; and
a pressing member disposed to generate a pushing force between the plunger and the connecting member when the plunger and the connecting member are adjacent to each other.
